(19) United States
(12) Patent Application Publication
(10) Pub. No.: US 2003/0207812 A1
(43) Pub. Date: Nov. 6, 2003

(54) Cd45 inhibitors

(76) Inventors: Marc Jerome Chapdelaine, Wilmington, DE (US); Katharine Knappenberger, Wilmington, DE (US); Gary Steelman, Wilmington, DE (US); Suzanne Suchard, Wilmington, DE (US); Linda Sygowski, Wilmington, DE (US); Rebecca Urbanek, Wilmington, DE (US); Chris Allan Veale, Wilmington, DE (US)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/168,758

(22) PCT Filed: Dec. 18, 2000
(86) PCT No.: PCT/GB00/04854

(51) Int. Cl. A61K 38/08; A61K 38/04; A61K 31/381; A61K 31/343; C07K 5/04; C07K 7/06; C07D 317/46; C07D 333/02

(52) U.S. Cl. 514/17; 514/438; 514/464; 514/469; 514/19; 514/567; 514/680; 514/677; 514/656; 530/329; 549/78; 549/434; 549/469; 552/292

(57) ABSTRACT

Substituted phenanthrene-9,10-diones in accord with structural diagram I,

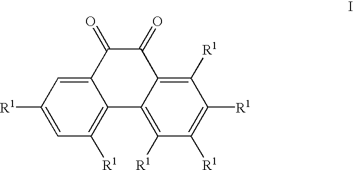

compositions thereof and methods for the use thereof, for the treatment of T cell mediated conditions such as autoimmune diseases and organ graft rejection. In compounds of the invention, R¹ at each occurrence is independently selected from hydrogen, halogen, NH-tosyl, N-di-tosyl, NH₂, NO₂, NH—CO—R², CO—NH—R², Ar, (CH₂)ₙCH(COOH)R³ COR³ and NHCOCH₂CH(COOH)NHR⁴, where R², R³ and R⁴ are a selected from a variety of substituted or unsubstituted alkyl and aryl groupstand oligopeptides.

### BACKGROUND

1. Field of the Invention

Compounds, compositions and methods for the treatment of immunologically-related diseases and disorders such as autoimmune disorders and organ graft rejection.

2. Related Art

Action of the immune system is known to be involved in immunologically-related diseases and disorders such as autoimmune disorders and in organ graft rejection (“OCR”). Hematopoietic, thymus-derived cells, (so-called “T cells”) have an important and pervasive role as regulators and effectors of the functions of the immune system. Hematopoietic cells, and T cells in particular have on their surfaces a major transmembrane glycoprotein designated CD45, characterized by a cluster of antigenic determinants. CD45 is also known as leukocyte common antigen (“LCA”). The cytosolic portion of CD45 has protein tyrosine phosphatase (“PTP”) activity and CD45 activity is known to be essential for TCR initiated T cell activation. Studies in CD45-deficient cell lines have shown that CD45 is a positive regulator of the T-Cell Receptor (“TCR”) and that CD45 functions in TCR regulation by dephosphorylating the src kinases p56^{lck} and p59^{fyn}). which allows autophosphorylation of the positive regulatory site on these enzymes; these reactions lead to downstream events and ultimately to T cell activation.

Available treatments for autoimmune disorders and OGR have therapeutic disadvantages. For example, Cyclosporin A, the drug most commonly used to treat OGR, has renal and CNS toxicity.

### SUMMARY OF THE INVENTION

Potent inhibitors of CD45 have been discovered. Such inhibitors are useful for the treatment of various autoimmune disorders as well as for treatment of OGR. Inhibition of the phosphatase activity of CD45 by compounds of the present invention has been shown by incubating the cytosolic portion of CD45 with the compounds and p-nitrophenyl phosphate (pNPP), a phosphatase substrate. Spectrophotometric monitoring has shown that the liberation of p-nitrophenol from the substrate by CD45 is inhibited in the presence of the compounds disclosed herein. Inhibition of the phosphatase activity of CD45 by compounds of the present invention has also been shown using a p56^{lck} carboxy-terminal phosphorylated peptide as a substrate. Compounds of the present invention have also been shown to inhibit proliferation of T cells in a T-cell proliferation assay.

Compounds of the present invention are substituted phenanthrene-9,10-diones in accord with structural diagram I:

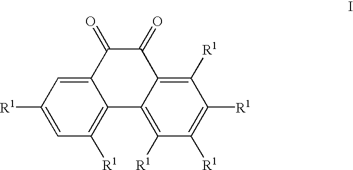

wherein R¹ at each occurrence is independently selected from hydrogen, halogen, NH₂, NO₂, NH—CO—R², CO—NH—R², Ar, (CH₂)ₙCH(COOH)R³ COR³, NHCOCH₂CH(COOH)NHR⁴ and N(R⁵)₂;

in compounds in accord with structural diagram I, R² is selected from (C₁-C₄)alkyl, (CH₂)ₙ,COOR⁶ and phenyl, wherein: n is an integer selected from the range 1 to 8; phenyl moieties of R² can be substituted at one, two or three positions with a moiety selected from halogen, NO₂ and CF₃; alkyl moieties of R² can be substituted with halogen, and R⁶ is selected from hydrogen and (C₁-C₄)alkyl;

in compounds in accord with structural diagram I where R¹ is Ar, Ar at each occurrence is independently selected from groups in accord with the following structural diagrams:

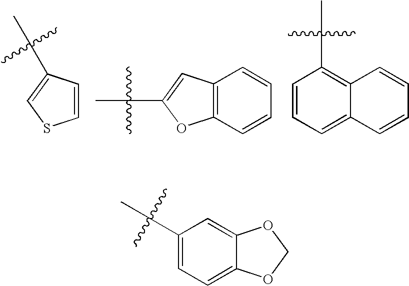

or from phenyl which can be substituted with a moiety selected from halogen, (C₁-C₄)alkyl, O-(C₁-C₄)alkyl and halo(C₁-C₄)alkyl;

in compounds in accord with structural diagram I where R¹ is COR³or (CH₂)ₙCH(COOH)R³, R³ at each occurrence is an N-linked oligopeptide selected from GQ-N-iBu, GQPQP, QQPQP, EQPQP, GEPQP, QEPQP, GQGQP, QQGQP, EQGQP, GEGQP, QEGQP, QQPEG, GQGEP, GQPQG, QQPQG, GEPQG, GQPEG, GGPEG, EGPEG, RGPEG, GEPEG, EEPEG, REPI G, GRPEG, ERPEG, RRPEG, GGPRG, EGPRG, RGPRG, GEPRG, EEPRG, REPRG, GRPRG, ERPRG and RRPRG;

in compounds in accord with structural diagram I where R¹ is NHCOCH₂CH(COOH)NHR⁴, R⁴ at each occurrence is a C-linked N-acetyl-amino acid, -tripeptide or -oligopeptide selected from Q, EGQ, ATEGQ, TATEGQ, and FTATEGQ;

in compounds in accord with structural diagram I where R¹ is N(R⁵)₂, R⁵ at each occurrence is selected from hydrogen and tosyl.

Particular compounds of the present invention within the scope of compounds in accord with structural diagram I are substituted phenanthrene-9,10-diones in accord with structural diagram II:

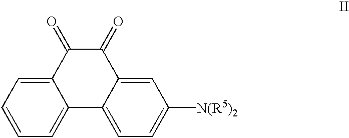

wherein R⁵ is selected from hydrogen and tosyl.

Further particular compounds of the present invention within the scope of compounds in accord with structural diagram I are substituted phenanthrene-9,10-diones in accord with structural diagram III:

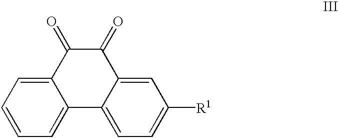

wherein R¹ is Ar and Ar selected from groups in accord with the following structural diagrams:

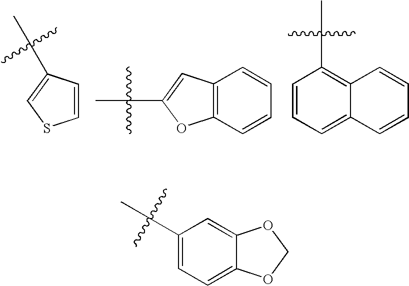

or from phenyl which can be substituted with a moiety selected from halogen, (C₁-C₄)alkyl, halo(C₁-C₄)alkyl and O-(C₁-C₄)alkyl.

Yet further particular compounds of the present invention within the scope of compounds in accord with structural diagram I are substituted phenanthrene-9,10-diones in accord with structural diagram IV:

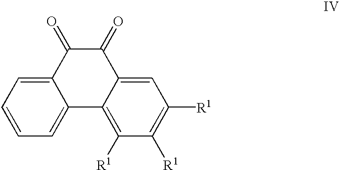

wherein R¹ is independently selected at each occurrence from hydrogen, halogen, NO₂, NH₂, (CH₂)ₙCH(COOH)R³ and COR³ where R³ is an N-linked peptide selected from GQ-N-iBu, GQPQP, QQPQP, EQPQP, GEPQP, QEPQP, GQGQP, QQGQP, EQGQP, GEGQP, QEGQP, QQPEG, GQGEP, GQPQG, QQPQG, GEPQG, GQPEG, GGPEG, EGPEG, RGPEG, GEPEG, EEPEG, REPEG, GRPEG, ERPEG, RRPEG, GGPRG, EGPRG, RGPRG, GEPRG, EEPRG, REPRG, GRPRG, ERPRG and RRPRG, with the proviso that no more than one R¹ moiety is other than hydrogen.

Other particular compounds of the present invention within the scope of compounds in accord with structural diagram I are substituted phenanthrene-9,10-dionies in accord with structural diagram V:

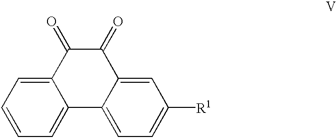

wherein R¹ is NHCOCH₂CH(COOH)NHR⁴, where R⁴ is a C-linked N-acetyl-oligopeptide selected from Q, EGQ, ATEGQ, TATEGQ, and FTATEGQ.

Still further particular compounds of the present invention within the scope of compounds in accord with structural diagram I are phenanthrene-9,10-diones which are not substituted at positions 6 and 8 and wherein R¹ is selected from hydrogen, NH₂, NO₂, and NHC(O)(CH₂)ₙCOOCH₃ where n is an integer selected from the range 1 to 8.

Compounds of the present invention are ligands of CD45 which, when bound, inhibit the activity of the protein tyrosine phosphatase (PTP) activity of the cytosolic portion of CD45. Binding of a compound of the present invention to CD45 inhibits the activity of CD45 essential for TCR initiated T cell activation. Thus, compounds of the invention inhibit the positive regulation of the TCR that leads to downstream events and T cell activation. Compounds of the present invention are useful to suppress the action of the immune system in immunologically-related diseases and disorders such as autoimmune disorders and organ graft rejection and to inhibit the action of T cells as functional regulators and effectors of the immune system.

The present invention also encompasses compositions made with compounds described herein useful for the treatment of immuniologically-related diseases and disorders and methods utilizing such compositions for treating such disorders.

### DETAILED DESCRIPTION OF THE INVENTION

As described herein, the ring atoms of 9,10-phenanthrenediones are identified with a conventional numbering system, in accord with the following structural diagram:

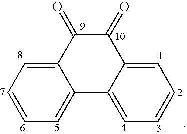

As described herein, amino acids (“AA”) of oligopeptides are identified by conventional abbreviations or one-letter code, as follows: Glycine/Gly/G; Glutamic acid/Glu/E; Glutamine/Gln/Q; Proline/Pro/P; Leucine/Leu/L; Arginine/Arg/R; Phenylalanine/Phe/F; Tyrosine/Tyr/Y, and Threonine/Thr/T.

As used herein, (C₁-C₄)alkyl has its conventionally-understood meaning and particularly means linear or branched hydrocarbon chains having from one to four carbon atoms and thus includes methyl, ethyl, propyl, isopropyl, n-butyl, sec-butyl, isobutyl, tert-butyl, and the like.

As used herein, halo(C₁-C₄)alkyl has its conventionally-understood meaning and particularly means (C₁-C4)alkyl as used herein wherein hydrogen atoms have been replaced by halogen atoms and thus includes monochloromethyl, trifluoromethyl, difluoroethyl, trifluoropropyl, perfluoro(C₁-C₄)alkyl, and the like.

As used herein perfluoro(C₁-C₄)alkyl has its conventionally-understood meaning and particularly means (C₁-C₄)alkyl as used herein wherein each hydrogen atom has been replaced by a fluorine atom and thus includes trifluoromethyl.

As used herein, (CH₂)ₙ has its conventionally-understood meaning and particularly means linear hydrocarbon chains having from one to n carbon atoms and thus includes methylene, ethylene, propylene, n-butylene groups, and the like.

As used herein, the terms halogen, halo, or halide have their conventionally-understood meanings and particularly mean chlorine, bromine, iodine or fluorine.

As used herein, the term tosyl has its conventionally-understood meaning and particularly means a group in accord with the following structural diagram:

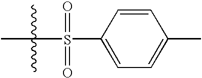

As used herein, the term “from the range 1 to 6” or the like, means any integral value in the stated range, in this example 1, 2, 3, 4, 5 or 6.

Definitions of Terms:

DMF, N,N-dimethylformamide; THF, tetrahydrofuran; HATU, ( )-(7-Azabenzotriazol-1-yl)-N, N, N′,N′-tetramethyluroniumhexafluorophosphate; TLC, thin-layer chromatography; NMR, nuclear magnetic resonance; TFA, trifluoroacetic acid; FMOC, N-(9-fluorenylmethoxycarbonyl); HPLC, high performance liquid chromatography; HRMS, high resolution mass spectroscopy; EDC, 1-(3-dimethylaminopropyl)-3-ethylcarbodiimide hydrochloride; DMAP, 4-dimethylaminopyridine; DMSO, dimethylsulfoxide; QTOF, quadropole time of flight; IC₅₀, concentration giving 50% inhibition; CC₅₀, concentration giving 50% cytotoxicity; AA, amino acid; ND, not determined.

HPLC method used: Analytical HPLC using an HP 1100 HPLC, with a C₁₈ Dynamax column (5 cm×4.6 mm, 3 μM particle size, 100 Å pore size), flow rate of 0.5 mL/min, 20%-60% CH₃CN in H₂O over 7.5 min, holding at 60% CH₃CN for 2.5 min, while monitoring at 254 and 210 nm.

HRMS method used: The sample was solubilized and diluted in MeOH. The QTOF mass spectrometer was calibrated using a PEG 400/600/1000 mixture solubilized in 50:50 acetonitrile/water with 50 mM ammonium acetate across a mass range 150-1150 Da. There was one sample submitted, with MW 1262 Da. This sample was run scanning the mass range 500-1300 Da using a calibration from the MW range 150-1300. The resolution for the instrument was measured at m/Δm=5100 at 50% peak height for the ion observed at m/z 953.1443. For each sample, 8 μL of solution was flow-injected into the mass spectrometer with a 35 μL/min flow of 80:20 acetonitrile/water (0.1% formic acid). A lock mass reference compound was infused into this flow at a rate of 2 μL/min (Ref. Compound exact mass 953.1443). Data was acquired from a single 3.5 minute analysis, and the signal was averaged from 0.9-1.8 min and then smoothed and centroided using a one-point lock mass correction using the reference compound listed above (m/z=953.1443).

### EXAMPLES

### Example 1

N-(9,10-Dioxo-9,10-dihydro-phenanthren-2-yl)-2-fluoro-benzamide:

To a solution of 2-amino-phenanthrene-9,10-dione (50 mg, 240 μmol) in THF (10 mL,) was added an excess of Na₂CO₃ (1g), followed by 2-fluorobenzoylchloride (46 μL, 384 μmol). The mixture was shaken overnight, then filtered and the solvent evaporated. The resulting material was purified on a silica gel column, using CH₂Cl₂-10% EtOAC/CH₂Cl₂ as the eluant to yield the pure amide N-(9,10-dioxo-9,10-dihydro-phenanthren-2-yl)-2-fluoro-benzamide as a red solid. ¹H NMR (300 MHz, DMSO-d₆) δ10.80 (1H, s), 8.45 (1H, d, J=2.4 Hz), 8.30 (1H, d, J=9 Hz), 8.23 (1H, d, J=8 Hz), 8.09 (1H, dd, J=2.3, 8.9 Hz), 8.02(1H, dd, J=1.3, 7.7 Hz), 7.80-7.70 (2H, m), 7.62 (1H, m), 7.50 (1H, dd, J=7.3, 7.3 Hz), 7.38 (2H, m); HPLC: 6.97 min.

The compounds of examples 2 to 8 inclusive were prepared by the method of Example 1, by utilizing the appropriate acid chloride.

### Example 2

2-Benzyloxy-N-(9,10-dioxo-9,10-dihydro-phenanthren-2-yl)-acetamide:

Mauve solid; ¹H NMR (300 MHz, DMSO-d₆) δ10.23 (1H, s), 8.38 (1H, d, J=2.1 Hz), 8.26 (1 H, d, J=9 Hz), 8.21 (1 H, d, J=7.8 Hz), 8.05-7.99 (2H, m),7.76 (1 H, dd, J=7.2, 7.2 Hz), 7.51-7.32 (6H, m), 4.65 (2H, s), 4.15 (2H, s); HPLC: 7.51 min.

### Example 3

N-(9,10-Dioxo-9,10-dihydro-phenanthren-2-yl)-butyramide:

Red solid; ¹H NMR (300 MHz, DMSO-d₆) δ10.25 (1H, s), 8.30 (1H, d, J=2.4 Hz), 8.23 (1H, d, J=8.4Hz), 8.19 (1H, d, J=7.2Hz), 7.98 (2H, m), 7.75 (1H, ddd, J=7.9,7.9, 1.3 Hz), 7.47 (1H, dd, J=7.2 Hz), 2.34 (2H, t, J=7.2 Hz), 1.64 (2H, tq, J=7.4, 7.4 Hz), 0.93 (3H, t, J=7.3 Hz); HPLC: 5.86 min.

### Example 4

N-(9,10-Dioxo-9,10-dihydro-phenanthren-2-yl)-2,2-dimethyl-propionamide:

Mauve solid; ¹H NMR (300 MHz, CDCl₃) δ8.41 (1H, dd, J=2.5, 8.7 Hz), 8.17 (1H, dd, J=1.3, 7.8 Hz), 7.98 (1 H, d, J=8.6 Hz), 7.94 (1H, d, J=7.2 Hz), 7.89 (1H, d, J=2.5 Hz), 7.70 (1H, ddd, J=1.4, 8,8 Hz), 7.54 (1H, br s), 7.44 (1H, dd, J=7.4, 7.4 Hz), 1.35 (9H, s); HPLC: 6.68 min.

### Example 5

N-(9,10-Dioxo-9,10-dihydro-phenanthren-2-yl)-2-nitro-benzamide:

Purple solid; ¹H NMR (300 MHz, DMSO-d₆) δ11.04 (1H, s), 8.39 (1H, d, J=2.4 Hz), 8.31 (1H, d, J 9Hz), 8.24(1H, d, J=8.1 Hz), 8.19 (1H, d, J=8.1 Hz), 8.04-7.99(2H, m), 7.86-7.75 (4H, m), 7.50 (1H, dd, J=7.5, 7.5 Hz); HPLC: 6.39 min.

### Example 6

2,4,6-Trichloro-N-(9,10-dioxo-9,10-dihydro-phenanthren-2-yl)-benzamide:

Red solid; ¹H NMR(300 MHz, ˜1:3 DMSO-d₆:CDCl₃) δ10.89 (1H,s), 8.38 (1H, d, J=1.8 Hz), 8.26 (1H, dd, J=2.4, 8.7 Hz), 8.10 (1H,dd, J=1.1, 7.6 Hz),8.07 (1H, d, J=8.7 Hz), 8.05 (1H, d,J=7.8 Hz), 7.75 (1H, ddd, J=1.6, 7.9, 7.9 Hz), 7.47 (3H,m);HPLC:8.47 min.

### Example 7

2,2,2-Trichloro-N-(9,10-dioxo-9,10-dihydro-phenanthren-2-yl)-acetamide:

Brown solid; ₁H NMR (300 MHz, CDCl₃) δ10.61 (1H, br s), 8.43 (1H, d, J=2.1 Hz), 8.35 (1H, dd, J=2.2, 9 Hz), 8.16 (1H, dd,J=1.2, 7.8 Hz), 8.07-8.01 (2H,m), 7.72 (1H, dd, J =7.5, 7.5 Hz), 7.47 (1H, dd, J=7.7, 7.7 Hz); HPLC: 7.65 min.

### Example 8

N-(9,10-Dioxo-9,10-dihydro-phenanthren-2-yl)-2-trifluoromethyl-benzamide:

Brown solid; ¹H NMR (300 MHz, ˜1:3 DMSO-d₆:CDCl₃) δ10.78 (1H, s), 8.43 (1H, d, J =2.2 Hz), 8.20 (1H, dd, J=2.5, 8.8 Hz). 8.10-8.06 (2H, m), 7.84-7.65 (6H, m), 7.46 (1H, dd, J=7.6, 7.6 Hz); HPLC: 7.37 min.

### Example 9

N-(9,10-Dioxo-9,10-dihydro-phenenathren-3-yl)-butyramide:

To a solution of 3-amino-phenanthrene-9,10-dione (50 mg. 240 μmol) in THF (10 mL) an excess of Na₂CO₃ (0.5 g) was added, followed by butyrylchloride (1 mL). The mixture was shaken overnight, then water (1 mL) was added and the mixture was shaken for an additional 30 min. The mixture was filtered through a pad of silica gel using THF as the eluant, and the eluate evaporated under reduced pressure to yield a solid. The solid products were triturated with Et₂O, and the residual solid material collected by vacuum filtration to yield the pure amide N-(9,10-dioxo-9,10-dihydro-phenanthren-3-yl)-butyramide as a brown solid. ¹H NMR (300 MHz, DMSO-d₆) δ10.44 (1H, s), 8.56 (1H, d, J=1.8 Hz),8.05-8.02 (3H,m), 7.84 (1H, ddd, J=1.5, 7.5, 7.5 Hz), 7.70 (1H, dd, J=1.8, 8.7 Hz), 7.56 (1H, dd, J=7.5, 7.5 Hz), 2.40 (2H, t, J=7.2 Hz), 1.66 (2H, tq, J=7.5, 7.5 Hz), 0.95 (3H, t, J=7.5 Hz); HPLC: 5.97 min.

The compounds of examples 10 to 13 inclusive were prepared by the method of Example 9, by utilizing the appropriate acid chloride.

### Example 10

N-(9,10-Dioxo-9,10-dihydro-phenenathren-3-yl)-2,2-dimethyl-propionamide:

Brown solid; ¹H NMR (300 MHz, DMSO-d₆) δ9.68 (1H, s), 8.60 (1H, s), 8.06-8.02 (3H, m), 7.9 0(1H, dd, J=1.8, 8.7 Hz), 7.84 (1H, ddd, J=1.2, 8.1, 8.1 Hz), 7.76 (1H, dd, J=7.5, 7.5 Hz), 1.29 (9H, s); HPLC: 6.89 min.

### Example 11

2,2,2-Trichloro-N(9,10-dioxo-9,10-dihydro-phenanthren-3-yl)-acetamide:

Brown solid; ¹H NMR (300 MHz, tfa shake-DMSO-d₆) δ8.62 (1H, d, J=1.9 Hz), 8.31 (1H, s), 8.11 (2 H, m), 7.96 (1H, dd, J=1.8, 8.7 Hz), 7.86 (1H, ddd, J=1.5, 7.8, 7.8 Hz); HPLC: 7.76 min.

### Example 12

N-(9,10-Dioxo-9,10-dihydro-phenanthren-3-yl)-2-fluoro-benzamide:

Yellow solid; ¹H NMR (300 MHz, DMSO-d₆) δ10.91 (1H, s), 8.64 (1H, s), 8.09-8.02 (3H, m), 7.89-7.82 (2H, m), 7.76 (1H, dd, J=7.5, 7.5 Hz), 7.68-7.57(2H, m),7.44-7.36(2H,m); HPLC: 7.15 min.

### Example13

N-(9,10-Dioxo-9,10-dihydro-phenanthren-3-yl)-2-trifluoromethyl-benzamide:

Brown solid; ¹H NMR (300 MHz, DMSO-d₆) δ11.10 (1H, s), 8.61 (1H, s), 8.09-7.99 (3H, m), 7.91-7.75 (6H, m), 7.57 (1H, dd, J=7.5, 7.5 Hz); HPLC: 7.36 min.

### Example 14

N-(9,10-Dioxo-9,10-dihydro-phenanthren-2-yl)- 4-methyl-N-[(4-methylphenyl)sulfonyl]-benzenesulfonamide:

To a solution of 2-amino-phenanthrene-9,10-dione (200 mg, 900 μmol) in CH₂Cl₂ (10 mL) under N₂, was added Et₃N (630 μL, 4.48 mmol), p-toluenesulfonyl chloride (TsCl, 340 mg, 1.79 mmol) and a catalytic amount of N,N-dimethylaminopyridine. The resultant solution was stirred overnight, after which time it was diluted with ethyl acetate (25 mL) and washed sequentially with saturated aqueous NH₄Cl, water and brine and then dried over Na₂SO₄. Filtration followed by evaporation under reduced pressure yielded a product which was purified by silica gel chromatography using CH₂Cl₂ as the eluant; the first material eluted from the column was the di-tosylate of 2-amino-phenanthrene-9,10-dione, N-(9,10-dioxo-9,10-dihydro-phenanthren-2-yl)-4-methyl-N-[(4-methylphenyl)sulfonyl]-benzenesulfonamide (39 mg, 73 μmol, 8 %) which was dried to a yellow solid. ¹H NMR (300 MHz, DMSO-d₆) δ8.39(1H, d , J=8.7 Hz), 8.31 (1H, d, J=8.0Hz), 8.06(1H, dd, J=1.2, 7.7 Hz), 7.81 (1H, dd, J=7.5, 7.5 Hz), 7.73 (4H, d, J=8.3 Hz), 7.60 (1H, dd, J=7.5, 7.5 Hz), 7.54 (1H, m), 7.52 (4H, d, J=8.3 Hz), 7.36 (1H, dd, J=2.4, 8.5 Hz), 2.50 (6H, s);HPLC: 9.85 min.

### Example 15

N-(9,10-Dioxo-9,10-dihydro-phenanthren-2-yl)-4-methyl-benzenesulfonamide:

A second material which eluted from the column was the mono-tosylate of 2-amino-9,10-phenenthrenedione, N-(9,10-dioxo-9,10-dihydro-phenanthren-2-yl)-4-methyl-benzenesulfonamide (53 mg, 140 mmol, 16%) which was dried to an orange solid. ¹H NMR (300 MHz, DMSO-d₆) δ10.77 (1H, s), 8.17 (1H, d, J=9 Hz), 8.12 (1H, d, J=8.1 Hz), 7.96 (1H, dd, J=1.5, 7.8 Hz), 7.78-7.29 (4H, m), 7.48 (1H, s), 7.46 (1H, t, J=8.4 Hz), 7.38 (2H, d, J=8.1 Hz), 2.32 (3H, s); HPLC: 7.14 min.

### Example 16

2-Benzofuran-2-yl-phenanthrene-9,10-dione:

To a mixture of 2-bromo-phenanthrene-9,10-dione (150 mg, 520 μmol) in dioxane (5 mL) and H₂ O (0.5 mL), benzo[b]furan-2-boronic acid (169 mg, 1.4 mmol), tris(dibenzylideneacetone)-dipalladium(0) (48 mg, 52 μmol), tri-o-tolylphosphine (32 mg, 1.4 mmol) and K₂CO₃ (220 mg, 1.57 mmol) were added. The resultant mixture was heated at 80° C. for 18 h and the solid material was removed by filtration. The soluble material was purified using a Rainin preparative HPLC on a Rainin phenyl column (25 cm×21.4 mm, 8 μM particle size, 60 Å, 40 mL/min, with 50% - 80% dioxane/H₂ O for 40 min, followed by 80%-50% for 5 min. If the purity at this stage was not sufficient, the material was then chromatographed on a silica gel column using CH₂-Cl₂ as the eluant. Purple solid; ¹H NMR (300 MHz, CDCl₃) δ8.64 (1H, d, J =1.8 Hz), 8.21 (2H, ddd, J=1.2, 7.8, 7.8 Hz), 8.10 (1H, d, J=8.7 Hz), 8.05 (1H, d, J=7.8 Hz), 7.75 (1H, dd, J=7.8 ,7.8 Hz), 7.64 (1H, d, J=7.5 Hz), 7.57 (1H, d, J=8.1 Hz), 7.50 (1H, dd, J=7.8, 7.8Hz), 7.35 (1H, ddd, J=1.2, 6.6, 6.6 Hz), 7.31-7.28(2H, m); HPLC: 10.97 min.

The compounds of examples 17 to 22 inclusive were prepared by the method of Example 16, by utilizing the appropriate boronic acid.

### Example 17

2-Thiophen-3-yl -phenanthrene-9,10-dione:

Orange solid; ¹H NMR (300 MHz, CDCl₃) δ8.40 (1H, d, J=2.1 Hz), 8.20 (1H, d, J=7.8 Hz), 8.04 (1H, d, J=8.1 Hz) 8.02 (1H, d, J=7.2 Hz), 7.94 (1H, dd, J=2.1, 8.4 Hz), 7.73 (1H, dd, J=7.5, 7.5 Hz), 7.64 (1H, m), 7.51-7.45 (3H, m); HPLC: 8.47 min.

### Example 18

2-(2-Fluoro-phenyl)-phenanthrene-9,10-dione:

Orange solid; ¹H NMR (300 MHz, CDCl₃) δ8.38 (1H, s), 8.23 (1H, d, J=7.8 Hz), 8.10 (1H, d, J=8.1 Hz), 8.07 (1H, d, J=8.1 Hz), 7.95 (1H,m), 7.75 (1H, dd, J=7.5,7.5 Hz), 7.56-7.51 (2H, m), 7.41-7.38 (1H, m), 7.28-7.20 (2H, m); HPLC: 8.76 min.

### Example 19

1 -Naphthalen-2-yl-phenanthrene-9,10-dione:

Orange solid; ¹H NMR (300 MHz, CDCl₃) δ8.35 (1H, d, J=2.1 Hz), 8.25 (1H, dd, J=1.2, 7.2 Hz), 8.16 (1H, d, J=8.4 Hz), 8.11 (1H, d, J=8.1 Hz), 7.96-7.84 (4H, m), 7.77 (1H, ddd, J=1.5, 7.7, 7.7 Hz), 7.52-7.47 (5H, m); HPLC: 10.31 min.

### Example 20

2-(2-Methoxy-phenyl)-phenanthrene-9,10-dione:

Orange solid; ¹H NMR (300 MHz, CDCl₃) δ8.36 (1H, d, J=1.9 Hz), 8.20 (1H, dd, J=1.4, 7.8 Hz), 8.06 (1H, s), 8.03 (1H, s), 7.93 (1H, dd, J=1.9, 8.3 Hz), 7.73 (1H, ddd, J=1.5, 7.6, 7.6 Hz), 7.47 (1H, ddd, J=0.6, 7.4, 7.4 Hz), 7.42-7.36 (2H, m), 7.10 (2H, m), 3.86 (3H, s); HPLC: 8.71 min.

### Example 21

2-Benzo[1,3]dioxol-5-yl-phenanthrene-9,10-dione:

Violet solid; ¹H NMR (300 MHz, CDCl₃) δ8.33 (1H, d, J=2.1 Hz), 8.20 (1H, dd, J=1.5, 7.8 Hz), 8.04 (1H, d, J=8.1 Hz), 8.02 (1H, d, J=7.2 Hz), 7.86 (1H, dd, J=1.8, 8.4 Hz), 7.73 (1H, ddd, J=1.5, 7.8, 7.8 Hz), 7.47 (1H, ddd, J=0.9, 7.4, 7.4 Hz), 7.21-7.14(2H, m), 6.91 (1H, d, J=7.8 Hz), 6.10 (2H, s); HPLC: 8.71 min.

### Example 22

2-(2-Ethoxy-phenyl)-phenanthrene-9,10-dione:

Orange solid; ¹H NMR (300 MHz, CDCl₃) δ8.43 (1H, s), 8.21 (1H, d, J=7.8 Hz), 8.04 (2H, m), 7.96 (1H, d, J=8.4 Hz), 7.73 (1H, dd, J=7.2, 7.2 Hz), 7.49-7.34 (3H, m), 7.12-6.99 (2H, m), 4.09 (2H, q, J=7.2 Hz), 1.39 (3H, t, J=6.9 Hz); HPLC: 9.55 min.

### Example 23

A Compound in Accord with Structural Diagram VII:

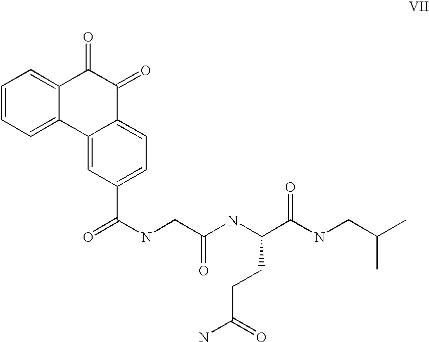

Polystyrene resin with the Ellman's aldehyde linker (50 mmol, see J. Org. Chem. 1997; 62, p. 1240) was swelled in DMF (1L) and acetic acid (10 mL) for 10 minutes, at which time isobutylamine (32 mL, 325 mmol) and sodium triacetoxyborohydride (69.5 g, 328 mmol) were added. The mixture was stirred with an overhead stirrer for two hours, then transferred to a fritted glass funnel and was washed with MeOH and DMF (1:1 mixture, 3×300 mL), DMF (3×300 mL), CH₂Cl₂ (5×300 ml) and methanol (5×300 ml). The resin was dried in vacuo at 40° C. for 16 h. The above resin (1.2 g, mmol) was swelled in DMF for 15 min, then washed three times with DMF. A solution of FMOCGln(Trt)OH (2.14 g, 3.5 mmol), HATU (1.14 g, 3 mmol), and diisopropylethylamine (1.1 mL, 6 mmol) in DMF (10 ml) was added to the resin and allowed to react for one hour. The liquid was drained and the resin was washed with DMF (10×10 mL). The resin was treated with 20% piperidine in DMF (10 mL) for 3 min, drained, then again treated with 20% piperidine in DMF (10 mL) for 7 min and drained. The resin was washed with DMF (10×10 mL). A mixture of FMOCGlyOH (1.04 g, 3.5 mmol). HATU (1.14 g, 3 mmol), and diisopropylethylamine (1.1 mL, 6 mmol) in DMF (10 mL) was added to the resin and allowed to react for 1 h. The liquid was drained and the resin was washed with DMF (10×10 mL). The resin was treated with 20% piperidine in DMF (10 mL) for 3 min, drained, then treated with another 20% piperidine in DMF (10 mL) for 7 min and drained. The resin was washed with DMF (10×10 mL). A mixture of 9,10-dioxo-9.10-dihydro-phenanthrene-3-carboxylic acid (880 mg, 3.5 mmol), HATU (1.14 g, 3 mmol), and diisopropylethylamine (1.1 mL, 6 mmol) was added to the resin and allowed to react for 1 h. The liquid was drained and the resin washed with DMF (10×10 mL), CH₂Cl₂ (5×10 mL), and Et₂O (5×10 mL). The resin was dried for 16 h in vacuo, then treated with TFA containing 2% water and 2% thioanisole (10 mL) for 2 h. The solids were removed by filtration and washed with CH₂Cl₂ (10 mL). The volume of combined filtrate was reduced to about 1 mL by rotary evaporation and precipitation was induced by addition of Et₂O (25 mL). The resulting solid was collected by vacuum filtration, washed with H₂ O (3×25 mL) and dried in vacuo for 2 h. This product was purified by preparative HPLC on a C₁₈ Dynamax column (21.4 mm×25 cm, 60 Å) using a gradient of 10% to 40% acetonitrile in water with 0.1% TFA. Fractions containing pure product were combined and lyophilized to give the compound in accord with structural diagram VII (170 mg) as an orange solid. Orange solid; HPLC: 3.95 min. Anal. Calcd. for C₂₆H₂₈N₄O₆-0.5H₂O-0.5CF₃CO₂H C, 58.06;H, 5.32; N, 10.03. Found: C, 57.98, 58.31; H, 5.48, 5.40; N, 9.88, 9.88.

### Example 24

A Compound in Accord with Structural Diagram VIII:

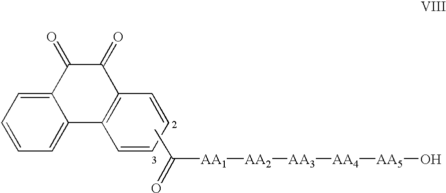

6-Chlorochlortrityl resin (6.19 g, 7 mmol) was swelled in dry CH₂Cl₂ (30 mL) and a solution of FMOCprolineOH (1.18 g, 3.5 mmol) and diisopropylethylamine (1.8 mL, 10.5 mmol) in dry CH₂Cl₂ (10 mL) was added. The reaction was gently mixed under a nitrogen atmosphere for 30 min, then MeOH (3 mL) was added to cap the unreacted sites. After 15 min, the resin was transferred to a fritted glass funnel and washed with DMF (2×50 mL), CH₂Cl₂ (2×50 mL). MeOH (2×50 mL), and Et₂O (2×50 mL). The resin was dried in vacuo at 50° C. to constant weight. A portion of this material (1.5 g, 0.25 mmol) was swelled in DMF (5 mL) for 15 min then washed with DMF (3×5 mL). The resin was treated with 20% piperidine in DMF (5 mL) for 3 min, drained, then treated with another 20% piperidine in DMF (5 mL) for 7 min and drained. The resin was washed with DMF (10×5 mL). A solution of FMOCglutamine(Trt)OH (610 mg, 1 mmol), HATU (380 mg, 1 mmol), and diisopropylethylamine (350 μL) in DMF (5 mL) was added to the resin and mixed with a gentle nitrogen bubbling for 2 h. The solution was drained and the resin was washed with DMF (10×5 mL). The resin was treated with 20% piperidine in DMF (5 mL) for 3 min, drained, then another portion of 20% piperidine in DMF (5 mL) was added and mixed for 7 min then drained. The resin was washed with DMF (10×5 mL). A solution of FMOCprolineOH (340 mg, 1 mmol), HATU (380 mg, 1 mmol), and diisopropylethylamine (350 μL) in DMF (5 mL) was added to the resin and mixed with a gentle nitrogen bubbling for 16 h. The solution was drained and the resin was washed with DMF (10×5 mL). The resin was treated with 20% piperidine in DMF (5 mL) for 3 min, drained, then another portion of 20% piperidine in DMF (5 mL) for 7 min and drained. The resin was washed with DMF (10×5 mL). A solution of FMOCglutamineOH (370 mg, 1 mmol), HATU (380 mg, 1 mmol), and diisopropylethylamine (350 μL) in DMF (5 mL) was added to the resin and mixed with a gentle nitrogen bubbling for 2.5 h. The solution was drained and the resin washed with DMF (10×5 mL). The resin was treated with 20% piperidine in DMF (5 mL) for 3 min, drained, then with another 20% piperidine in DMF (5 mL) for 7 min and drained. The resin was washed with DMF (10×5 mL). A solution of FMOCglutamineOH(OtBu) (830 mg, 1 mmol), HATU (380 mg, 1 mmol), and diisopropylethylamine (350 μL) in DMF (5 mL,) was added to the resin and mixed with a gentle nitrogen bubbling for 2 h. The solution was drained and the resin was washed with DMF (10×5 mL). The resin was treated with 20% piperidine in DMF (5 mL) for 3 min, drained, then with another 20% piperidine in DMF (5 mL) for 7 min and drained. The resin was washed with DMF (10×5 mL). A solution of 9,10-dioxo-9,10-dihydro-phenanthrene-3-carboxylic acid (250 mg, 1 mmol), HATU (380 mg, 1 mmol), and diisopropylethylamine (350 μL) in DMF (5 mL) was added to the resin and mixed with a gentle nitrogen bubbling for 16 h. The solution was drained and the resin was washed with DMF (10×5 mL), CH₂Cl₂ (5×5 mL), Et₂O (5×5mL), then dried under a stream of N₂. The resin was treated with TFA containing 2% thioanisole and 2% water (5 mL) for 3 h. The solids were filtered off and the filtrate was evaporated to a red oil. Addition of Et₂O (10 mL) induced precipitation, and this solid was collected by vacuum filtration to give the product (300 mg) as a pink solid. The material was purified by preparative HPLC on a C₁₈ Dynamax column (21.4 mm×25 cm, 60 Å) using a gradient of 10% to 26% acetonitrile in water with 0.1% TFA. Fractions containing pure product were combined and lyophilized to give M374187 (100 mg) as a yellow solid. HPLC: 2.55 min; Anal. Calcd. for C₄₀H₄₅N₇O₁₃-1.0H₂O-0.5CF₃CO₂H C, 54.30; H, 5.28; N, 10.81; Found C, 54.10, 54.04; H, 5.19, 5.23;N, 10.99, 11.01.

The compounds shown in tables 1 and 2, below, were synthesized by the method of Example 24, using either 9,10-dioxo-9,10-dihydro-phenanthrene-3-carboxylic acid or 9,10-dioxo-9,10-dihydro-phenanthrene-2-carboxylic acid as the starting material and a suitable peptide fragment:

Table 1 shows compounds of the invention in accord with structural diagram VIII, having 3-carboxy-linked peptides.

TABLE 1

| Example No. | AA₁ | AA₂ | AA₃ | AA₄ | AA₅ |
|---|---|---|---|---|---|
| 24 | Glu | Gln | Pro | Gln | Pro |
| 25 | Gly | Gln | Pro | Gln | Pro |
| 26 | Gln | Gln | Pro | Gln | Pro |
| 27 | Gly | Glu | Pro | Gln | Pro |
| 28 | Gln | Glu | Pro | Gln | Pro |
| 29 | Gly | Gln | Gly | Gln | Pro |
| 30 | Gln | Gln | Gly | Gln | Pro |
| 31 | Glu | Gln | Gly | Gln | Pro |
| 32 | Gly | Glu | Gly | Gln | Pro |
| 33 | Gln | Glu | Gly | Gln | Pro |
| 34 | Gln | Gln | Pro | Glu | Gly |
| 35 | Gly | Gln | Gly | Glu | Pro |
| 36 | Gly | Gln | Pro | Gln | Gly |
| 37 | Gln | Gln | Pro | Gln | Gly |
| 38 | Gly | Glu | Pro | Gln | Gly |
| 39 | Gly | Gln | Pro | Glu | Gly |

### Example 25

Yellow solid; HPLC: 2.67 min; Anal. Calc. For C₃₇H₄₁N₇O₁₁-1.5H₂O-0.4CF₃CO₂H C,54.54; H, 5.38N, 11.78. Found: C, 54.52; 54.42; H, 5.40; 5.32;N, 11.72; 11.80.

### EXAMPLE 26

Yellow solid; HPLC: 2.41 min.

### EXAMPLE 27

Yellow solid; HPLC: 2.88 min; Anal. Calcd. for C₃₇H₄₀N₆O₁₂-1.0OH₂O-0.5CF₃CO₂H C, 54.61; H, 5.13 N, 10.05. Found C, 54.82, 54.81; H, 5.51, 5.52; N, 10.01; 10.01.

### EXAMPLE 28

Yellow solid; HPLC: 2.60 min; Anal. Calcd. for C₄₀H₄₅N₇O₁₃-1.5H₂O-0.6CF₃CO₂H C, 53.37; H, 5.28; N, 10.57. Found C, 53.49; 53.43; H, 5.14, 5.15; N,10.85, 10.82.

### Example 29

Yellow solid; HPLC: 2.34 min; Anal. Calcd. for C₃₄H₃₇N₇O₁₁-0.7H₂O-0.6CF₃CO₂H C, 52.80; H, 4.91; N, 12.24. Found C, 52.59, 52.81; H, 4.92, 4.90; N, 12.53, 12.57.

### Example 30

Yellow solid; HPLC: 2.07 min; Anal. Calcd. for C₃₇H₄₂N₈O₁₂-2.0H₂O-0.6CF₃CO₂H C, 51.25; H, 5.25; N, 12.52. Found C, 51.31, 51.39; H, 4.97, 4.97; N, 12.75, 12.79.

### Example 31

Yellow solid; HPLC: 2.47 min: Anal. Calcd. for C₃₇H₄₁N₇O₁₃-1.0H₂O-0.5CF₃CO₂H C, 52.66; H, 5.06; N. 11.31. Found C, 52.79, 52.78; H, 5.17, 5.20; N, 11.31, 11.31.

### Example 32

Yellow solid; HPLC; 2.52 min; Anal. Calcd. for C₃₄H₃₆N₆O₁₂-0.5H2O-0.5CF₃CO₂1H C. 53.44; H, 4.80; N, 10.69. Found 53.29, 53.24; H, 4.96, 4.98; N, 10.73, 10.74.

### Example 33

Yellow solid; HPLC: 2.25 min; Anal. Calcd. for C₃₇H₄₁N₇O₁₃-1.0H₂O-0.5CF₃CO₂H C, 52.66; H, 5.06; N, 11.31. Found C, 52.59, 52.61; H, 5.17, 5.18; N, 11.46, 11.37.

### Example 34

Yellow solid; HPLC: 2.13min; Anal. Calcd. for C₃₇H₄₁N₇O₁₃-1.5H₂O-0.5CF₃CO₂H C, 52.11; N, 5.12; N, 11.20. Found C, 51.94, 51.98; H, 4.93, 4.93; N. 11.42, 11.42.

### Example 35

Yellow solid: HPLC: 2.49 min; Anal. Calcd. for C₃₄H₃₆N₆O₁₂-1.5H₂O-0.5CF₃CO₂H C, 52.24; H. 4.95: N, 10.44. Found C, 51.87, 51.91; H, 4.93, 4.86; N, 10.56, 10.57.

### Example 36

Yellow solid. HPLC; 2.26min; Anal. Calcd. for C₃₄H₃₇N₇O₁₁-1.0H₂O-0.5CF₃CO₂H C, 52.92, 53.08; H, 5.01, 5.00; N, 12.60, 12.66.

### Example 37

Yellow solid; HPLC: 2.02 min; Anal. Calcd. for C₃₇H₄₂N₈O₁₂-1.0H₂O-0.6CF₃CO₂H C, 52.30; H, 5.12; N, 12.77. Found C, 52.35, 52.28; H, 5.21, 5.15; N, 13.08, 13.05.

### EXAMPLE38

Yellow solid; HPLC: 2.50 min; Anal. Calcd. for C₃₄H₃₆N₆O₁₂-0.5H₂O-0.5CF₃CO₂H C, 53.4; H, 4.80; N, 10.68. Found C, 53.48, 53.48; H, 4.80, 4.85; N, 10.84, 10.88.

### Example 39

Yellow solid; HPLC: 2.47 min; Anal. Calcd. for C₃₄H₃₆N₆O₁₂-1.0H₂O-0.5CF₃CO₂H C, 52.83; H, 4.87; N, 10.56. Found C, 52.99, 53.30; H, 4.80, 4.83; N, 10.87, 10.84.

Table 2 shows compounds of the invention in accord with structural diagram VIII, having 2-carboxy-linked peptides.

TABLE 2

| Example No. | AA₁ | AA₂ | AA₃ | AA₄ | AA₅ |
|---|---|---|---|---|---|
| 40 | Gly | Gly | Pro | Glu | Gly |
| 41 | Glu | Gly | Pro | Glu | Gly |
| 42 | Arg | Gly | Pro | Glu | Gly |
| 43 | Gly | Glu | Pro | Glu | Gly |
| 44 | Glu | Glu | Pro | Glu | Gly |
| 45 | Arg | Glu | Pro | Glu | Gly |
| 46 | Gly | Arg | Pro | Glu | Gly |
| 47 | Glu | Arg | Pro | Glu | Gly |
| 48 | Arg | Arg | Pro | Glu | Gly |
| 49 | Gly | Gly | Pro | Arg | Gly |
| 50 | Glu | Gly | Pro | Arg | Gly |
| 51 | Arg | Gly | Pro | Arg | Gly |
| 52 | Gly | Glu | Pro | Arg | Gly |
| 53 | Glu | Glu | Pro | Arg | Gly |
| 54 | Arg | Glu | Pro | Arg | Gly |
| 55 | Gly | Arg | Pro | Arg | Gly |
| 56 | Glu | Arg | Pro | Arg | Gly |
| 57 | Arg | Arg | Pro | Arg | Gly |

### Example 40

Yellow solid: HPLC; 2.77 min; Anal. Calc. For C₃₁H₃₁N₅O₁₁-1.0OH₂O-0.5CF₃CO₂H C, 53.04; H, 4.66; N, 9.66. Found: C, 53.10, 52.99; H, 4.68, 4.61; N, 9.87, 9.93.

### Example 41

Yellow solid; HPLC: 2.83 min; Anal. Calc. For C₃₄H₃₅N₅O₁₃ -0.5H₂O-0.5CF₃CO₂H C, 53.37; H, 4.67; N, 8.89. Found: C, 53.35, 53.46; H, 4.75, 4.83; N, 8.85, 8.89 min.

### Example 42

Yellow solid; HPLC: 2.74 min; Anal. Calc. For C₃₅H₄₀N₈O₁₁-0.5H₂O-1.5CF₃CO₂H C, 49.14; H, 4.61; N, 12.06. Found: C, 49.07, 49.12; H, 4.64, 4.70; N, 12.06, 12.08.

### Example 43

Yellow solid; HPLC: 2.78 min; Anal. Calc. For C₃₄H₃₅N₅O₁₃-0.75H₂O-0.4CF₃CO₂H C, 53.53; H, 4.76; N, 8.97. Found: C, 53.75, 53.75; H, 4.80, 4.79; N, 9.04, 9.06.

### Example 44

Yellow solid; HPLC: 2.83 min; Anal. Calc. For C₃₇H₃₉N₅O₁₅-0.5H₂O-0.5CF₃CO₂H C, 53.09; H, 4.75; N, 8.15. Found: C, 52.92, 52.86; H, 4.94, 4.88; N, 8.22, 8.22.

### Example 45

Yellow solid; HPLC: 2.74 min; Anal. Calc. For C₃₈H₄₄N₈O₁₃-1.5H₂O-1.4CF₃CO₂H C, 48.64; H, 4.84; N, 11.12. Found: C, 48.36, 48.46; H, 4.68, 4.71; N, 11.23, 11.28.

### Example 46

Yellow solid; HPLC: 2.49 min; Anal. Calc. For C₃₅H₄₀N₈O₁₁-0.5H₂O-1.5CF₃CO₂H C, 49.14; 4.61; N, 12.06. Found: C, 49.00, 48.67; H, 4.68, 4.67; N, 12.24, 12.14.

### Example 47

Yellow solid; HPLC: 2.83 min; Anal. Calc. For C₃₈H₄₄N8O₁₃-1.0H₂O-1.5CF₃CO₂H C, 48.76; H, 4.74; N, 11.10. Found: C, 48.69, 48.96; H, 4.74, 4.76; N, 11.33, 11.34.

### Example 48

Yellow solid; HPLC: 2.48 min; Anal. Calc. For C₃₉H₄₉N₁₁O₁₁-2.0H₂O-2.2CF₃CO₂H C, 45.94; H, 4.90; N, 13.58. Found: C, 45.72, 45.90; H, 4.61, 4.62; N, 13.75, 13.79. C, 45.94; H, 4.90; N, 13.58. Found: C, 45.72 ,45.90; H, 4.61, 4.62; N, 13.75, 13.79.

### Example 49

Yellow solid; HPLC: 2.74 min; Anal. Calc. For C₃₂H₃₆N₈O₉-0.5H₂O-1.5CF₃CO₂H C, 49.07; H, 4.53; N, 13.08. Found: C, 49.06, 49.10; H, 4.53, 4.54; N, 13.30, 13.29.

### Example 50

Yellow solid; HPLC: 2.84 min; Anal. Calc. For C₃₅H₄₀N₈O₁₁-0.5H₂O-1.5CF₃CO₂H C, 49.14; H, 4.61; N, 12.06. Found: C, 48.87, 49.00; H, 4.63, 4.64; N, 12.23, 12.29.

### Example 51

Yellow solid; HPLC: 2.49min; Anal. Calc. For C₃₆H₄₅N₁₁O₉-1.5H₂O-2.5CF₃CO₂H C, 45.27; H, 4.68; N, 14.16. Found: ,. 45.10, 45.14; H, 4.60, 4.55; N, 14.27, 14.28.

### Example 52

Yellow solid; HPLC: 2.81 min; Anal. Calc. For C₃₅H₄₀N₈O₁₁-1.25H₂O-1.5CF₃CO₂H C, 48.44; H, 4.71; N, 11.89. Found: C, 48.40, 48.28; N. 4.62, 4.60; N, 12.13, 12.10.

### Example 53

Yellow solid; HPLC: 2.92 min; Anal. Calc. For C₃₈H₄₄N₈O₁₃-1.0H₂O-1.5CF₃CO₂H C, 48.76; H, 4.74; N, 11.10. Found: C, 48.36, 48.52; H, 4.67, 4.69; N, 11.20, 11,26.

### Example 54

Yellow solid; HPLC: 2.56 min; Anal. Calc. For C₃₉H₄₉N₁₁O₁₁-1.5H₂O-2.3CF₃CO₂H C, 46.05; H, 4.81; N, 13.55. Found: C, 45.83, 45.90; H, 4.63, 4.63; N, 13.79, 13.73.

### Example 55

Yellow solid; HPLC: 2.57 min; Anal. Calc. For C₃₆H₄₅N₁₁O₉-2.0H₂O-2.2CF₃CO₂H C, 45.66; H, 4.86; N, 14.50. Found: C, 45.46, 45.58; H, 4.57, 4.58; N, 14.57, 14.61.

### Example 56

Yellow solid; HPLC: 2.71 min; Anal. Calc. For C₃₉H₄₉N₁₁O₁₁-1.5H₂O-2.3CF₃CO₂H C, 46.05; H, 4.81; N, 13.55. Found: C, 46.03, 45.83; H, 4.68, 4.65; N, 13.74, 13.72.

### Example 57

Yellow solid; HPLC: 2.09 min; Anal. Calc. For C₄₀H₅₄N₁₄O₉-2.5H₂O-3.2CF₃CO₂H C, 43.37; H, 4.89; N, 15.26. Found: C, 43.40, 43.28; H, 4.57, 4.52; N, 15.38, 15.34.

### Example 58

A Compound in Accord with Structural Diagram IX:

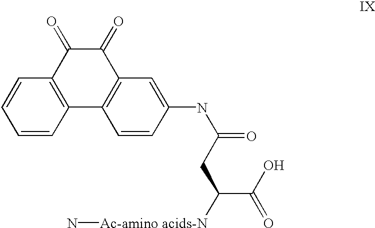

A compound in accord with structural diagram IX having the N-Ac-amino acid sequence N-Ac-Glu-Gly-Gln was synthesized as follows: FMOCAsp(OH)OtBu (3.3 g, 8 mmol) and N-methylmorpholine (900 μL, 8 mmol) were dissolved in dry THF (20 mL) and chilled to −10° C. Isobutylchloroformate (910 μL, 7 mmol) was added dropwise, maintaining reaction temperature below −7° C. To this mixture was added a suspension of 2-amino-phenanthrene-9,10-dione (1.12 g, 5 mmol) in dry THF (40 mL), and the reaction was allowed to warm to ambient temperature. The disappearance of 2-aminophenanthrene-9,10-dione was monitored by TLC (R_{f}=0.42, 25:75 EtOAc:CH₂Cl₂, v/v on a silica gel plate), and reaction was complete after 7 d. The solvent was removed by rotary evaporation and the residue was partitioned between EtOAc and sat'd. aq. NH₄Cl. The organic phase was washed with sat'd. aq. NaHCO₃ and brine, then dried over anhydrous MgSO₄. This mixture was filtered and the filtrate was concentrated to a red-brown solid and chromatographed on silica gel with 15:85, EtOAC:CH₂Cl₂, v/v. This gave FMOCAsp-2-aminophenanthrene-9,10-dione (1.92 g, 3.1 mmol, 63%) as a red solid. This intermediate was dissolved in CH₂Cl₂(100 mL) and TFA (30 mL) was added; after 1h the volatiles were removed by rotary evaporation. Residual TFA was removed by dissolving the material in CH₂Cl₂ and concentration on the rotary evaporator twice. This was dissolved in dry CH₂Cl₂ (100 mL), diisopropylethylamine (5 mL) and dry DMF (10 mL) were added under N₂. 2-chlorochlortrityl resin (4.5 g of 1.2 mmol/g, 5.5 mmol) was added to the mixture and mixed by shaking. After 3 h, MeOH (10 mL) was added and the shaking was continued for an additional 10 min. The resin was collected by vacuum filtration and washed with CH₂Cl₂ (5×10 mL), DMF (2×10 mL), CH₂Cl₂ (2×10 mL) and MeOH (3×10 mL). The resin was dried for 64 h in vacuo. An incorporation of 2.3 mmol of FMOCAsp(3-aminophenanithrene-9,10-dione) was calculated based on the increase of mass to 5.8 g. The resin was swelled in DMF (25 mL) for 1 h, then washed with DMF (5×25 mL). The resin was treated with 20% piperidine in DMF (10 mL) for 3 min, drained, then was treated with more 20% piperidine in DMF (10 mL) for 7 min and drained. The resin was washed with DMF (10×10 mL). A solution of FMOCGln(trt)OH (6.72 g, 11 mmol), HATU (3.8 g, 10 mmol), and diisopropylethylamine (3.5 mL) in DMF (15 mL) was added to the resin and mixed with gentle nitrogen bubbling for 2 h. The solution was drained and the resin was washed with DMF (10×10 mL). The resin was treated with 10 ml 20% piperidine in DMF (10 mL) for 3 min, drained, then another portion of 20% piperidine in DMF (10 mL) was added for 7 min and drained. The resin was washed with DMF (10×10 mL). A portion of the resin (ca. 10%) was removed for other reactions and the remaining resin was treated with a solution of FMOCGlyOH (1.93 g, 11 mmol), HATU (3.8 g, 10 mol), diisopropylethylamine (3.5 mL) in DMF (15 mL) for 1 h. The reaction was drained and washed with DMF (10×10 mL). The resin was treated with 20% piperidine in DMF (10 mL) for 3 min, drained, then treated with additional 20% piperidine in DMF (10 mL) for 7 min and drained. The resin was washed with DMF (10×10 mL). A portion of the resin (about 10%) was removed for other reactions and the remaining resin was treated with a solution of FMOCGlu(tBu)OH (4.68 g, 11 mmol). HATU (3.8 g, 10 mmol). and diisopropylethylamine (3.5 mL) in DMF (15 mL) for 16 h. The solution was drained and the resin was washed with DMF (10×10 mL). The resin was treated with 20% piperidine in DMF (10 mL) for 3 min, drained, then additional 20% piperidine in DMF (10 mL) was added for 7 min and drained. The resin was washed with DMF (10×10 mL). A portion of this material (ca. 15%) was removed and treated with a solution of Ac₂O (1 mL) and diisopropylethylamine (2 mL) in DMF (5 mL) for 2 h. The solution was drained and washed withDMF (10×5 mL), CH₂Cl ₂ (5×5 mL), Et₂O (5×5 mL) and dried under a N₂ stream. The resin was treated with a solution of TFA containing 2% H₂O and 2% thioanisole (5 mL) for 2 h. The solids were removed and the solution was concentrated to a red oil. Addition of Et₂O (10 mL) induced precipitation of a red solid, which was collected by vacuum filtration. The material was purified by preparative HPLC on a C₁₈ Dynamax column (21.4 mm×25 cm, 60 Å) using a gradient of 10% to 26% acetonitrile in H₂O with 0.1% TFA. Fractions containing pure product were combined and lyophilized to give the product (40 mg) as a red solid. HPLC: 2.69 min; HRMS theor. [M+H]:695.2313 amu; obs. [M+H] 695.2307 amu; deviation of −0.9 ppm.

Table 3 shows compounds of the invention in accord with structural diagram IX, made by the method of Example 58, utilizing the appropriate peptide fragments.

TABLE 3

| Example No. | amino acid sequence |
|---|---|
| 58 | N—Ac-Glu-Gly Gin |
| 59 | N—Ac-Gln |
| 60 | N—Ac-Ala-Thr-Glu-Gly-Gln |
| 61 | N—Ac-Thr-Ala-Thr-Glu-Gly-Gln |
| 62 | N—Ac-Phe-Thr-Ala-Thr-Glu-Gly-Gln |

### Example 59

Red Solid; HPLC: 2.85 min; HRMS theor. [M+H]: 509.1672 amu; obs. [M+H] 509.1657 amu; Deviation of −3 ppm.

### Example 60

Red Solid; HPLC: 2.69 min; HRMS theor. [M+H]: 867.3161 amu; obs. [M+H] 867.3145 amu; Deviation of −1.8 ppm.

### Example 61

Red Solid; HPLC: 2.69 min; HRMS theor. [M+H]: 968.3638 amu; obs. [M+H] 968.3619; Deviation of −1.9 ppm.

### Example 62

Red Solid; HPLC: 3.81 min; HRMS theor. [M+H]: 1115.4322 amu; obs. [M+H] 1115.4286 amu; Deviation of −3.2 ppm.

### Example 63

N-(9,10-Dioxo-9,10-dihydro-phenanthren-4-yl)-succinamic Acid Methyl Ester:

Using a BOHDAN Automated RAM Synthesizer; to a solution 4-amino-phenanthrene-9,10-dione (50 mg, 238 μmol) in THF (10 mL) an excess of Na₂CO₃ (1 g) was added, followed by 3-carbomethoxypropionyl chloride (381 μmol; 323 μL). The reaction cycle, which consisted of vortexing the mixture twice for 10 s, was repeated 10 times, and this was followed by filtering and evaporating the solvent. The reaction mixture was then purified on a 10 g silica gel column, using CH₂Cl₂-5% EtOAC/CH₂Cl₂-10% EtOAC/CH₂Cl₂-20% EtOAC/CH₂Cl₂ as the eluant, to yield the pure N-(9,10-Dioxo-9.10-dihydro-phenanthren-4-yl)-succinamic acid methyl ester as a brown solid. ¹H NMR (300 MHz, DMSO-d₆) δ10.26 1H, s), 8.43 (1H, d, J=7.8 Hz), 8.01 (1H, dd, J=1.4, 7.8 Hz), 7.92 (1H, dd, J=1.4, 7.4 Hz). 7.75 (1H, ddd, J=1.5, 7.8, 7.8 Hz), 7.68 (1H, dd, J=1.5, 7.8 Hz), 7.53 (1H, d, J=7.8 Hz), 7.50 (1H, d, J=7.5 Hz), 3.62 (3H, d), 2.67 (4H, m); HPLC: 3.57 min.

The compounds of examples 64 to 79 inclusive were prepared by the method of Example 63, utilizing the appropriate acid chloride.

### Example 64

7-(5-Nitro-9,10-dioxo-9,10-dihydro-phenanthren-2-ylcarbamoyl)-heptanoic Acid Methyl Ester:

Brown solid; ¹H NMR (300 MHz, DMSO-d₆) δ10.37 (1H, s), 8.39 (1H, d, J=2.4 Hz), 8.22 (1H, dd, J=1.5, 8.0 Hz), 8.11 (1H, dd, J=1.2, 8.3 Hz), 7.85 (1H, dd, J=2.7, 9.0 Hz), 7.66 (1H, dd, J=7.8, 7.8 Hz), 7.38 (1H, d, J=8.6 Hz), 3.58 (3H, s), 2.38-2.27 (4H, m), 1.62-1.48 (4H, m), 1.30 (4H, m); HPLC: 7.48 min.

### Example 65

4-(5-Nitro-9,10-dioxo-9,10-dihydro-phenanthren-2-ylcarbamoyl)-butyric Acid Methyl Ester:

Red Solid; ¹H NMR (300 MHz, DMSO-d₆) δ10.41 (1H, s), 8.39 (1H, d, J=2.4 Hz), 8.22 (1H, d, J=1.1, 7.8 Hz), 8.11 (1H, dd, J=1.2, 8.0 Hz), 7.84 (1H, dd, J=2.6, 9.0Hz), 7.66 (1H, dd, J=7.9, 7.9 Hz), 7.38 (1H, d, J=9.0 Hz), 3.60 (3H, s), 2.42 (2H, t, J=7.4 Hz), 2.39 (2H, t, J=7.4 Hz), 1.86 (2H, tt, J=7.7, 7.7 Hz); HPLC: 5.96 min.

### Example 66

N-(5-Nitro-9,10-dioxo-9,10-dihydro-phenanthren-2-yl)-succinamic Acid Methyl Ester:

Red Solid; ¹H NMR (300 MHz, DMSO-d₆ ) δ10.51 (1H, s), 8.39 (1H, d, J=2.3 Hz), 8.22 (1H, dd, J=0.8, 7.9 Hz), 8.11 (1H, dd, J=1.1, 7.9 Hz), 7.82 (1H, dd, J=2.3, 8.7 Hz), 7.66 (1H, dd, J=7.6, 7.6 Hz), 7.38 (1H, d, J=8.6 Hz), 3.61 (3H, s), 2.66 (4H, m); HPLC: 5.58 min.

### Example 67

4-(9,10-Dioxo-9,10-dihydro-phenanthren-4-ylcarbamoyl)-butyric Acid Methyl Ester:

Orange solid; ¹H NMR (300 MHz, DMSO-d₆ ) δ10.18 (1H, s), 8.35 (1H, d, J=9 Hz), 7.99 (1H, d, J=6 Hz), 7.93 (H, d, J=9 Hz), 7.75-7.67 (2H, s), 7.53 (1H, d, J=6 Hz), 7.50 (1H, d, J=9 Hz), 3.62 (3H, s), 2.44-2.37 (4H, m), 1.91-1.82 (2H, m), HPLC: 3.89 min.

### Example 68

7-(9,10-Dioxo-9,10-dihydro-phenanthren-4-ylcarbamoyl)-heptanoic Acid Methyl Ester:

Orange solid; ¹H NMR (300 MHz, DMSO-d₆ ) δ10.14 (1H, s), 8.35 (1H, d, J=8.0 Hz), 7.99 (1H, d, J=7.6 Hz), 7.92 (1H, d, J=7.7 Hz), 7.75-7.66 (2H, m), 7.51 (2H, m), 3.59 (3H, s), 2.37 (2H, t, J=7.2 Hz), 2.31 (2H, t, J=7.6 Hz), 1.58 (4H, m), 1.33 (4H, m); HPLC: 5.81 min.

### Example 69

9-(9,10-Dioxo-9,10-dihydro-phenanthren-4-ylcarbamoyl)-nonanoic Acid Methyl Ester:

Brown solid; ¹H NMR (300 MHz; DMSO-d₆ ) δ10.12 (1H, s), 8.35 (1H, d, J=8.5 Hz), 7.99 (1H, d, J=7.7 Hz), 7.92 (1H, d, J=7.6 Hz), 7.70 (2H, m), 7.52 (1H, d, J=7.6 Hz), 7.50 (1H, d, J=7.7 Hz), 3.58 (3H, s), 2.37 (2H, t, J=7.0 Hz), 2.29 (2H, t, J=7.5 Hz), 1.61 (2H, m), 1.52 (2H, m), 1.29 (8H, m); HPLC: 7.31 min.

### Example 70

4-(9,10-Dioxo-9,10-dihydro-phenanthren-2-ylcarbamoyl)-butyric Acid Methyl Ester:

Brown solid; ¹H NMR (300 MHz, DMSO-d₆ ) δ10.27 (1H, s), 8.28 (1H, d, J=2.5 Hz), 8.23 (1H, d, J=8.9 Hz), 8.19 (1H, d, J=8.1 Hz), 7.99 (1H, d, J=7.3 Hz), 7.94 (1H, d, J=2.0 Hz), 7.75 (1H, dd, J=7.7, 7.7 Hz), 7.47 (1H, dd, J=7.5, 7.5 Hz), 3.60 (3H, s), 2.40 (4H, m), 1.87 (2H, tt, J=7.4, 7.4 Hz); HPLC: 5.58 min.

### Example 71

7-(9,10-Dioxo-9,10-dihydro-phenanthren-2-ylcarbamoyl)-heptanoic Acid Methyl Ester:

Brown solid; ¹H NMR (300 MHz, DMSO-d₆ ) δ10.22 (1H, s), 8.28 (1H, d, J=2.4 Hz), 8.20 (2H, m), 7.98 (2H, m), 7.75 (1H, dd, J=7.3, 7.3 Hz), 7.47 (1H, dd, J=7.7, 7.7 Hz), 3.30 (3H, s), 2.37-2.28 (4H, m), 1.63-1.52 (4H, m), 1.31 (4H, m); HPLC: 7.26 min.

### Example 72

9-(9,10-Dioxo-9,10-dihydro-phenanthren-2-ylcarbamoyl)-nonanoic Acid Methyl Ester:

Brown solid; ¹H NMR (300 MHz, DMSO-d₆ ) δ10.22 (1H, s), 8.28 (1H, d, J=2.0 Hz), 8.22 (1H, d, J=8.9 Hz), 8.19 (1H, d, J=8.2 Hz), 7.98 (2H, m), 7.75 (1H, dd, J=7.3, 7.3 Hz), 7.47 (1H, dd, J=7.7, 7.7 Hz), 3.57 (3H, s), 2.34 (2H, t, J=7.2 Hz), 2.28 (2H, t, J=7.3 Hz), 1.61 (2H, m), 1.52 (2H, m); HPLC: 8.57 min.

### Example 73

N-[5-(3-Methoxycarbonyl-propionylamino)-9,10-dioxo-9,10-dihydro-phenanthren-2-yl]-succinamic Acid Methyl Ester:

Pink solid; ¹H NMR (300 MHz, DMSO-d₆ ) δ10.40 (1H, s), 10.18 (1H, s), 8.39 (1H, d, J=8.7 Hz), 8.23 (1H, d, J=2.0 Hz), 7.90 (2H, m), 7.63 (1H, d, J=7.9 Hz), 7.45 (1H, dd, J=7.7, 7.7 Hz), 3.62 (3H, s), 3.61 (3H, s), 2.65 (8H, m); HPLC: 3.64 min.

### Example 74

4-[5-(4-Methoxycarbonyl-butyrylamino)-9,10-dioxo-9,10-dihydro-phenanthren-2-ylcarbamoyl]-butyric Acid Methyl Ester:

Brown solid; ¹H NMR (300 MHz, DMSO-d₆ ) δ10.31 (1H, s), 10.11 (1H, s), 8.29 (1H, d, J=8.8 Hz), 7.91 (2H, m), 7.64 (1H, d, J=7.2 Hz), 7.47 (1H, dd, J=7.7, 7.7 Hz), 3.63 (3H, s), 3.60 (3H, s), 2.40 (8H, m), 1.87 (4H, m); HPLC: 4.24 min.

### Example 75

7-[5-(7-Methoxycarbonyl-heptanoylamino)-9,10-dioxo-9,10-dihydro-phenanthren-2-ylcarbamoyl]-heptanoic Acid Methyl Ester:

Brown solid; ¹H NMR (300 MHz, DMSO-d₆ ) δ10.23 (1H, s), 10.03 (1H, s), 8.29 (1H, d, J=8.9 Hz), 8.20 (1H, s), 7.90 (2H, m), 7.63 (1H, d, J=8.1 Hz), 7.44 (1H, dd, J=7.7, 7.7 Hz), 3.58 (6H, s), 2.31 (8H, m), 1.56 (8H, m), 1.30 (8H, m); HPLC: 7.09 min.

### Example 76

9-[5-(9-Methoxycarbonyl-nonanoylamino)-9,10-dioxo-9,10-dihydro-phenanthren-2-ylcarbamoyl]-nonanoic Acid Methyl Ester:

Brown solid; ¹H NMR (300 MHz, DMSO-d6 ) δ10.22 (1H, s), 10.03 (1H, s), 8.29 (1H, d, J=8.6 Hz), 8.20 (1H, s), 7.93 (1H, dd, J=2.0, 8.9 Hz), 7.89 (1H, d, J=7.6 Hz), 7.62 (1H, d, J=7.6 Hz), 7.44 (1H, dd, J=7.7, 7.7 Hz), 3.57 (6H, s), 2.38-2.26 (8H, m), 1.62-1.50 (8H, m), 1.28 (16H, m); HPLC: 9.21 min.

### Example 77

4-(9,10-Dioxo-9,10-dihydro-phenanthren-3-ylcarbamoyl)-butyric Acid Methyl Ester:

Orange solid: ¹H NMR (300 MHz, DMSO-d₆ ) δ10.46 (1H, s), 8.53 (1H, d, J=1.7 Hz), 8.04-7.98 (3H, m), 7.83 (1H, dd, J=7.3, 7.3 Hz), 7.67 (1H, dd, J=1.5, 8.8 Hz), 7.56 (1H, dd, J=7.2, 7.2 Hz), 3.61 (3H, s), 2.45 (4H, m), 1.89 (2H, tt, J=7.7, 7.7 Hz); HLPC: 5.68 min.

### Example 78

8-(9,10-Dioxo-9,10-dihydro-phenanthren-3-ylcarbamoyl)-heptanoic Acid Methyl Ester:

Orange solid; ¹H NMR (300 MHz, DMSO-d₆ ) δ10.42 (1H, s), 8.54 (1H, d, J=1.6 Hz), 8.04-7.98 (3H, m), 7.83 (1H, ddd, J=1.2, 7.6, 7.6 Hz), 7.68 (1H, dd, J=1.4, 8.6 Hz), 7.56 (1H, dd, J=7.4, 7.4 Hz), 3.58 (3H, s), 2.40 (2H,t, J=7.3 Hz), 2.30 (2H, t, J=7.3 Hz), 1.63 (2H, m), 1.54 (2H, m), 1.32 (4H, m); HPLC: 7.44 min.

### Example 79

9-(9,10-Dioxo-9,10-dihydro-phenanthren-3-ylcarbamoyl)-nionanoic Acid Methyl Ester:

Brown solid; ¹H NMR (300 MHz, DMSO-d₆ ) δ10.42 (1H, s), 8.54 (1H, s), 8.02 (3H, m), 7.84 (1H, dd, J=7.7, 7.7 Hz), 7.69 (1H, d, J=8.7 Hz), 7.56 (1H, dd, J=7.7, 7.7 Hz), 3.57 (3H, s), 2.40 (2H, t, J=7.3 Hz), 2.28 (2H, t, J=7.3 Hz), 1.63 (2H, m), 1.52 (2H, m), 1.29 (8H, m); HPLC: 8.82 min.

Compounds of examples 80 and 81 were made as follows: To a solution of 2-amino-phenanthrene-9,10-dione (670 mg, 3.0 mmol) in THF (70 mL) diisopropylethylamine (1.1 mL, 6.0 mmol) was added, followed by methyl 4-chloro-4-oxobutyrate for Example 81 N-(9,10-dioxo-9,10-dihydro-phenanthren-2-yl)-succinamic acid methyl ester (490 μL, 4.0 mmol) or acetyl chloride for Example 82, N-(9,10-dioxo-9,10-dihydro-phenanthren-2-yl)-acetamide (284 μL, 4.0 mmol), Each reaction was stirred for 2.5 h, then the THF was removed by rotary evaporation, and the reaction mixture was chromatographed on silica gel (9:1, CH₂Cl₂: MeOH, v/v) and the product eluted.

### Example 80

N-(9,10-Dioxo-9,10-dihydro-phenanthren-2-yl)-succinamic Acid Methyl Ester:

Brown solid; ¹H NMR (300 MHz, DMSO-d₆ ) δ10.39 (1H, s), 8.29 (1H, d, J=2.3 Hz), 8.23 (1H, d, J=9.1 Hz), 8.19 (1H, d, J=8.0 Hz),7.99 (1H, dd, J=1.4, 7.8 Hz), 7.92 (1H, dd, J=2.3, 8.7 Hz), 7.74 (1H, ddd, J=1.1, 7.7, 7.7 Hz), 7.47 (1H, dd, J=7.1, 7.1 Hz), 3.61 (3H, s), 2.65 (4H, m); HPLC: 4.61 min.

### Example 81

N-(9,10-Dioxo-9,10-dihydro-phenanthren-2-yl)-acetamide:

Purple solid; ¹H NMR (300 MHz, DMSO-d₆ ) δ10.33 (1H, s), 8.26 (1H, d, J=2.7 Hz), 8.23 (1H, d, J=9.1 Hz), 8.19 (1H, d, J=8.0 Hz), 7.99 (1H, dd, J=1.1, 7.7 Hz), 7.95 (1H, dd, J=2.7, 8.5 Hz), 7.75 (1H, ddd, J=1.6, 8.0, 8.0 Hz), 7.47 (1H, dd, J=7.2, 7.2 Hz), 2.10 (3H, s); HPLC: 4.42 min.

### Example 82

N-[7-(3-Methoxycarbonyl-propionylamino)-9,10-dioxo-9,10-dihydro-phenanthren-2-yl]-succinamic Acid Methyl Ester:

The compound of example 82 was synthesized by a method that differed from that of example 80 only in that double the amount of acid chloride, methyl 4-chloro-4-oxobutyrate, was used and 2,7-diamino-phenanthrene-9,10-dione was used in place of 2-amino-phenanthrene-9,10-dione. Purple solid; ¹H NMR (300 MHz, DMSO-d₆ ) δ10.34 (2H, s), 8.25 (2H, d, J=1.9 Hz), 8.10 (2H, d, J=9.0 Hz), 7.89 (2H, dd, J=1.7, 8.4 Hz), 3.61 (6H, s), 2.50 (8H, m); HPLC: 4.78 min.

### Example 83

2,7-Diamino-phenanthrene-9,10-dione:

2,7-Dinitro-phenanthrene-9,10-dione (3.0 g, 10 mmol) was suspended in THF (200 mL) and Raney Nickel (ca. 1 g) was added. The reaction was hydrogenated on a Parr shaker under 50 psi H₂ for 2.5 h, at which time the mixture was filtered through a plug of diatomaceous earth and the filtrate was evaporated under rotary evaporation. The product was chromatographed on silica gel (15% to 100% EtOAc in CH₂Cl₂ as eluant) and dried to afford 2,7-diamino-phenanthrene-9,10-dione (1.1 g, 4.6 mmol, 46%) as a black solid. ¹H NMR (300 MHz, TFA-d₁-DMSO-d₆ ) δ8.77 (2H, d, J=2.7 Hz), 8.76 (2H, d, J=9.1 Hz), 8.62 (2H, dd, J=2.7, 9.1 Hz), Anal. Calcd for C₁₄H₆N₂O₆ C, 56.39; H, 2.02; N, 9.39 Found: C, 56.20, 56.44; H, 2.17, 2.14; N, 8.89, 8.95.

### Examples 84 and 85

Compounds of examples 84 and 85 were made by the following method: 2,5-dinitro-phenanthrene-9,10-dione (3.0 g, 10 mmol) was dissolved in THF (250 mL) and Raney Nickel (ca. 0.3 g) was added. The reaction was hydrogenated on a Parr shaker under 50 psi H₂ until the uptake of H₂ slowed (ca. 5 h). The reaction mixture was filtered through celite and the filtrate evaporated by rotary evaporation. The reaction mass was a mixture of 2-amino-5-nitro-phenanthrene-9,10-dione and 2,5-diamino-phenanthrene-9,10-dione, and was chromatographed on silica gel.

The compound of example 84, 2-amino-5-nitro-phenanthrene-9,10-dione was eluted with EtOAc:CH₂Cl₂ 1:4, and dried to afford a black solid (400 mg, 1.7 mmol, 17%), ¹H NMR (300 MHz, DMSO-d₆ ) δ8.11 (1H, dd, J=1.4, 8.1 Hz), 7.99 (1H, dd, J=1.1, 8.1 Hz), 7.47 (1H, dd, J=7.5, 7.5 Hz), 7.25 (1H, d, J=2.8 Hz), 7.08 (1H, d, J=8.6 Hz), 6.82 (1H, dd, J=2.8, 8.6 Hz), 6.18 (2H, s).

The compound of example 85, 2,5-diamino-phenanthrene-9,10-dione was eluted with EtOAc and dried to afford a black solid (1.2 g, 5 mmol, 50%), ¹H NMR (300 MHz, DMSO-d₆) δ8.20 (1H, d, J=8.7 Hz), 7.25 (1H, dd, J=1.5, 7.2 Hz), 7.15 (1H, d, J=2.5 Hz) 7.12-7.01 (2H, m), 6.88 (1H, dd, J=2.5, 8.5 Hz), 5.71 (2H, s), 5.46 (2H, s); HPLC: 1.89 min.

The compounds of Examples 86 to 89 inclusive were prepared by the method of Example 83 using the appropriate nitrated 9,10-phenanthrenedione as the starting material.

### Example 86

1-Amino-phenanthrene-9,10-dione:

Purple solid; ¹H NMR (300 MHz, TFA-d₁-DMSO-d₆) δ8.25 (1H, d, J=8.2 Hz), 8.05 (1H, dd, J=1.2, 7.8 Hz), 7.77 (1H, ddd, J=1.5, 7.2, 8.6 Hz), 6.97 (1H, dd, J=2.1, 7.4 Hz); HPLC: 5.13 min.

### Example 87

2-Amino-phenanthrene-9,10-dione:

Black solid; ¹H NMR (300 MHz, DMSO-d₆) δ8.00 (1H, d, J=7.8 Hz), 7.92-7.87 (2H, m), 7.65 (1H, ddd, J=1.7, 8.0, 8.0 Hz), 7.31 (1H, dd, J=7.0, 7.0Hz), 7.19 (1H, d, J=2.8 Hz), 6.91 (1H, dd, J=2.5, 8.5 Hz), 5.87 (2H, s), Anal. Calcd for C₁₄H₉NO₂-0.33 H₂O C, 73.35 4.25 N, 6.11. Found: C, 73.33, 73.39; H, 4.22, 4.21; N, 6.28, 6.29.

### Example 88

3-Amino-phenanthrene-9,10-dione:

Brown solid; ¹H NMR (300 MHz, DMSO-d₆) δ7.99 (2H, m), 7.83 (1H, d, J=8.4 Hz), 7.78 (1H, d, J=7.2 Hz), 7.53 (1H, dd, J=7.2, 7.2 Hz), 7.30 (1H, s), 6.89 (2H, s), 6.64 (1H, dd, J=1.5, 8.6 Hz), Anal. Calcd, For C₁₄H₉O₂N-0.10 H₂O C, 74.72; H, 4.12; N, 6.22 Found: C, 74.94, 74.74; H, 4.10, 4.08; N, 6.26, 6.30.

### Example 89

4-Amino-phenanthrene-9,10-dione:

Black solid; ¹H NMR (300 MHz, DMSO-d₆) δ8.56 (1H, d, J=8.0 Hz), 7.94 (1H, dd, J=1.4, 7.8 Hz), 7.73 (1H, ddd, J=1.6, 7.8, 7.8 Hz), 7.41 (1H, ddd, J=1.0, 7.9, 7.9 Hz), 7.36 (1H, dd, J=3.2, 5.9 Hz), 7.20 (2H, m), 5.84 (2H, s), Anal. Calcd. For C_{14H}₉NO₂ C, 75.33; H, 4.06; N, 6.27 Found: C, 75.12, 75.12; H, 4.28,4.29; N, 6.21, 6.20.

### Example 90

N-(9,10-Dioxo-9,10-dihydro-phenanthren-3-yl)-succinamic Acid Methyl Ester:

To a solution of 3-amino-phenanthrene-9,10-dione (400 mg, 1.8 mmol) in dioxane (100 mL) was added a large excess of Na₂CO₃ followed by methyl 4-chloro-4-oxobutyrate (240 μL, 2.0 mmol). The reaction was stirred for several hours, then filtered and the solvent was removed under reduced pressure. The material was chromatographed on silica gel with 20% EtOAc in CH₂Cl₂ as eluant and dried to give N-(9,10-Dioxo-9,10-dihydro-phenanthren-3-yl)-succinamic acid methyl ester as an orange solid. ¹H NMR (300 MHz, DMSO-d₆) δ10.60 (1H, s), 8.55 (1H, d, J=1.5 Hz), 8.04-7.98 (3H, m), 7.84 (1H, ddd, J=1.6, 7.9, 7.9 Hz), 7.65 (1H, dd, J=2.0, 8.7 Hz), 7.56 (1H, dd, J=7.6, 7.6 Hz), 3.62 (3H, s), 2.72 (2H, m), 2.67 (2H, m); HPLC: 5.30 min.

### Example 91

N-(9,10-Dioxo-9,10-dihydro-phenanthren-1-yl)-succinamic Acid Methyl Ester:

To a solution of 1-amino-phenanthrene-9,10-dione (50 mg, 240 μmol) in THF (50 mL) was added Na₂CO₃ (210 mg, 2.2 mmol), followed by methyl 4-chloro-4-oxobutyrate (60 μL, 500 μmol). The mixture was stirred overnight filtered, and diluted with ethyl acetate. The organic layer was washed with water, brine, dried over Mg₂SO₄, filtered, and chromatographed on silica gel with ethyl acetate as the eluant to yield N-(9,10-dioxo-9,10-dihydro-phenanthren-1-yl)-succinamic acid methyl ester as an orange solid. ¹H NMR (300 MHz, DMSO-d₆) δ12.04 (1H, s), 8.60 (1H, d, J=7.7 Hz), 8.32 (1H, d, J=7.7 Hz), 8.03 (2H, m), 7.79 (2H, m), 7.56 (1H, dd, J=7.2, 7.2 Hz), 3.61 (1H, dd, J=7.2 Hz), 2.81 (2h, m) 2.69 (2H, m); HPLC: 6.49 min.

### Examples 92 to 95

Compounds of examples 92 to 95 inclusive were prepared substantially in accordance with the procedures disclosed in Schmidt, J. Chem. Ber. 1903, 23, 3726-3730, taking into account the results disclosed by Ray, F. E.; Francis, W. C. J. Org. Chem. 1943, 8, 52-59, which procedures and results are incorporated herein by reference.

### Example 92

2-Nitro-phenanthrene-9,10-dione:

Orange solid; ¹H NMR (300 MHz, DMSO-d₆) δ8.80 (1H, d, J=2.8 Hz), 8.69 (1H, d, J=8.5 Hz), 8.60 (1H, dd, J=2.4, 8.7 Hz), 8.51 (1H, d, J=8.31 Hz), 8.22 (1H, dd, J=1.4, 7.7 Hz), 7.93 (1H, ddd, J=1.6, 8.1, 8.1 Hz), 7.73 (1H, dd, J=7.5, 7.5 Hz). Anal. Calcd. For C_{14H}₇NO₄: C, 66.41; H, 2.79; N. 5.53; Found: C, 66.70, 66.71; H, 2.83, 2.84; N. 5.57, 5.63.

### Example 93

4-Nitro-phenanthrene-9,10-dione:

Orange solid; ¹H NMR (300 MHz, DMSO-d₆) δ8.27 (1H, dd, J=1.5, 7.9 Hz), 8.17 (1H, dd, J=1.3, 7.9 Hz), 8.10 (1H, dd, J=1.5, 7.8 Hz), 7.79-7.71 (2H, m), 7.63 (1H, ddd, J=0.9, 7.7, 7.7 Hz), 7.46 (1H, d, J=8.0 Hz), Anal. Calcd. For C₁₄H₇NO₄-0.25 H₂O C, 65.25; H, 2.93; N, 5.43. Found: C, 65.19, 65.24; H, 2.82. 2.83; N, 5.43, 5.44.

### Example 94

2,7-Dinitro-phenanthrene-9,10-dione:

Yellow solid; ¹H NMR (300 MHz, tfashake-DMSO-d₆) δ8.77 (2H, d, J=2.7 Hz), 8.76 (2H, d, J=9.1 Hz), 8.62 (2H, dd, J=2.7, 9.1 Hz). Anal. Calcd. for C₁₄H₆N₂O₆ C, 56.39; H, 2.02; N, 9.39 Found: C, 56.20, 56.44; H, 2.17, 2.14; N, 8.89, 8.95.

### Example 95

2,5-Dinitro-phenanthrene-9,10-dione:

Yellow solid; ¹H NMR (300 MHz, DMSO-d₆) δ8.67 (1H, d, J=2.5 Hz), 8.54 (1H, dd, J=2.5, 9.1 Hz), 8.36 (1H, dd, J=1.5, 8.1 Hz), 8.26 (1H, dd, J=1.3, 7.8 Hz), 7.87 (1H, dd, J=7.8, 7.8 Hz), 7.73 (1H, d, J=8.8 Hz),

### Example 96

2-Bromo-phenanthrene-9,10-dione:

This compound was prepared substantially in accordance with the procedures disclosed in Bhatt, M. V.; Tetrahedron 1964, 20, 803-821, which procedures are incorporated herein by reference. Orange solid; ¹H NMR (300 MHz, DMSO-d₆) δ8.30 (1H, d, J=7.9 Hz), 8.27 (1H, d, J=8.6 Hz), 8.07 (1H, d, J=2.4 Hz), 8.04 (1H, d, J=1.0, 7.6 Hz), 7.96 (1H, dd, J=2.7, 8.9 Hz), 7.79 (1H, ddd, J=1.7, 8.2, 8.2 Hz), 7.56 (1H, dd, J=7.6, 7.6 Hz), Anal. Calcd. For C₁₄H₇O₂ C, 58.57; H, 2.46. Found: C, 58.22, 58.99; H, 2.63, 2.65.

### Examples 97 and 98

The compounds of examples 97 and 98 were prepared substantially in accordance with the procedures disclosed in Langenbeck, W.; Schaller, R.; Arneberg, K. Chem. Ber. 1942, 75, 1483-1488, which procedures are incorporated herein by reference.

### Example 97

9,10-Dioxo-9,10-dihydro-phenanthrene-2-carboxylic Acid:

Yellow solid; ¹H NMR (300 MHz, DMSO-d₆) δ13.45 (1H, s), 8.51 (1H, d, J=1.9 Hz), 8.46 (1H, d, J=8.2 Hz), 8.37 (1H, d, J=8.0 Hz), 8.24 (1H, dd, J=2.0, 8.2 Hz), 8.08 (1H, dd, J=0.75, 7.6 Hz), 7.83 (1H, dd, J=7.2, 7.2 Hz), 7.61 (1H, dd, J=7.5, 7.5 Hz) Anal calcd. For C₁₅H₈O₄-0.4 H₂O C, 69.45; H, 3.42 Found: C, 69.59, 69.66; H 3.16, 3.17.

### Example 98

9,10-Dioxo-9,10-dihydro-phenanthrene-3-carboxylic Acid:

Orange solid; ¹H NMR (300 MHz, DMSO-d₆) δ13.66 (1H, br s), 8.71 (1H, s), 8.35 (1H, d, J=8.0 Hz), 8.12 (1H, d, J=8.0 Hz), 8.08-8.02 (2H, m), 7.81 (1H, dd, J=7.5, 7.5 Hz), 7.58 (1H, dd, J=7.5, 7.5 Hz), Anal. Calcd. For C₁₅H₈O₄-0.2 H₂O C, 70.42; H, 3.31 Found: C, 70.61, 70.76; H, 3.41, 3.44.

### Example 99

3-Nitro-phenanthrene-9,10-dione:

This compound was prepared substantially in accordance with the procedures disclosed in Braithwaite, R. S. W.; Holt, P. F. J. Chem. Soc. 1959, 2304-2305, which procedures are incorporated herein by reference. Orange solid; ¹H NMR (300 MHz, TFA-d₁-DMSO-d₆) δ9.02 (1H, d, J=1.7 Hz), 8.50 (1H, d, J=8.1 Hz), 8.29 (2H, m), 8.13 (1H, dd, J=1.1, 7.6 Hz), 7.85 (1H, ddd, J=1.3, 7.7, 7.7 Hz), 7.64 (1H, dd, J=7.4, 7.4 Hz), Anal. Calcd. For C₁₄H₇NO₄-0.1H₂O: C, 65.94; H, 2.85; N, 5.49. Found: C, 65.78, 66.02; H, 2.92, 2.93; N, 5.41, 5.40.

### Examples 100 and 101

The compounds of examples 100 and 101 were prepared substantially in accordance with the procedures disclosed in Mosettig, E.; Van de Kamp, J. J. Am. Chem. Soc. 1930, 52, 3704-3710, which procedures are incorporated herein by reference.

### Example 100

3-Acetyl-phenanthrene-9,10-dione:

Orange solid; ¹H NMR (300 MHz, DMSO-d₆) δ8.70 (1H, d, J=2.4 Hz), 8.45 (1H, d, J=8.1 Hz), 8.13 (1H, d, J=8.1 hz), 8.07 (1H, dd, J=2.4, 7.8 Hz), 8.02 (1H, dd, J=1.5, 8.1 Hz), 7.82 (1H, ddd, J=2.4, 7.6, 7.6 Hz), 7.58 (1H, ddd, J=1.2, 7.5, 7.5 Hz), 2.75 (3H, s); HPLC: 5.48 min.

### Example 101

2-Acetyl-phenanthrene-9,10-dione:

Yellow solid; ¹H NMR (300 MHz, DMSO-d₆) δ8.49-8.45 (2H, m), 8.39 (1H, d, J=8.1 Hz), 8.27 (1H, dd, J=2.1, 8.4 Hz), 8.08 (1H, dd, J=1.2, 7.8 Hz), 7.83 (1H, ddd=1.5, 7.4, 7.4 Hz), 7.61 (1H, dd, J=7.5 Hz), 2.68 (3H, s); HPLC: 5.27 min.

### Example 102:

To a solution of 9,10-dioxo-9,10-dihydro-phenanthrene-3-carboxylic acid (1.1 g, 4.5 mmol) in anhydrous DMF (10 mL) under N₂ was added t-butyl glycine hydrochloride (760 mg, 4.6 mmol), EDC (1.1 g, 6.0 mmol), DMAP (60 mg, 500 μmol) and diisopropylethylamine (2.1 mL, 12 mmol) and the resultant solution was stirred for 16 h. The reaction was diluted with EtOAc and washed with 1M HCI, water, sat'd aq. NaHCO₃ and brine. The organic layer was dried over MgSO₄, filtered, concentrated, and chromatographed on silica gel (3:1 EtOAc:CH₂Cl₂, v/v) to afford a yellow solid (500 mg). This solid was subsequently recrystallized from refluxing EtOAc to afford pure [(9,10-dioxo-9,10-dihydro-phenanthrene-3-carbonyl)-amino]-acetic acid tert-butyl ester (310 mg, 31%). Yellow solid; ¹H NMR (300 MHz, DMSO-d₆) δ9.29 (1H, dd, J=6.1, 6.1 Hz), 8.71 (1H, s), 8.38 (1H, d, J=8.0 Hz), 8.13 (1H, d, J=8.3 Hz), 8.07 (1H, dd, J=1.5, 7.9 Hz), 7.97 (1H, dd, J=1.5, 8.3 Hz), 7.85 (1H, ddd, J=1.5, 7.8, 7.8 Hz), 7.58 (1H, ddd, J=7.2, 7.2 Hz), 4.00 (2H, d, J=6.1 Hz), 1.45 (9H, s); Anal. Calcd. For C₂₁H₉₁NO₅-0.2H₂O: C, 68.36; H, 5.30; N, 3.80. Found: C, 68.22, 68.58 ; H, 5.17, 5.20; N, 3.87, 3.89.

The compound of example 103 was prepared by the method of Example 102, utilizing 9,10-dioxo-9,10-dihydro-phenanthrene-2-carboxylic acid as the starting material.

### Example 103

[(9,10-Dioxo-9,10-dihydro-phenanthrene-2-carbonyl)-amino]-acetic acid tert-butyl ester:

Yellow solid; 1H NMR (300 MHz, DMSO-d₆) δ9.23 (1H, dd, J=5.7, 5.7 Hz), 8.54 (1H, d, J=1.9Hz), 8.46 (1H, d, J=8.7Hz), 8.39 (1H, d, J=7.9 Hz), 8.22 (1H, dd, J=2.3, 8.7 Hz), 8.07 (1H, dd, J=1.6, 7.7 Hz). 7.82 (1H, ddd, J=1.5, 7.9, 7.9 Hz). 7.59 (1H, dd, J=7.4, 7.4 Hz), 3.94 (2H, d, J=6.3 Hz), 1.44 (9H, s); Anal. Calcd. For C₂₁H₁₉NO₅-0.1 H₂O: C, 68.69; H, 5.27; N, 3.81. Found: C, 68.67, 68.84; H, 5.38, 5.41; N, 3.77, 3.78.

### Example 104

To a solution of [(9,10-dioxo-9,10-dihydro-phenanthrene-3 -carbonyl)-amino]-acetic acid tert-butyl ester (200 mg, 550 μmol) in CH₂Cl₂ (10 mL) under N₂ was added TFA (10 mL). This mixture was stirred for 2 h and concentrated under reduced pressure. The residue was dissolved in CH₂Cl₂ and evaporated once again to rid residual TFA. The material was recrystallized from refluxing EtOAc to afford [(9,10-dioxo-9,10-dihydro-phenanthrene-3-carbonyl)-amino]-acetic acid (70 mg, 47%). Orange solid; ¹H NMR (300 MHz, DMSO-d₆) δ12.7 (1H, s), 9.29 (1H, dd, J=5.7, 5.7 Hz), 8.71 (1H, s), 8.39 (1H, d, J=7.9 Hz), 8.13 (1H, d, J=8.3Hz), 8.07 (1H, dd, J=1.2, 7.5 Hz), 7.98 (1H, dd, J=1.6, 8.2Hz), 7.85 (1H, ddd, J=1.3, 8.7, 8.7 Hz), 7.59 (1H, dd, J=7.5, 7.5 Hz), 4.03 (2H, d, J=5.6 Hz); Anal. Calcd. For C₁₇H₁₁NO₅-1.0H₂O: C, 62.39; H, 4.00; N, 4.28. Found: C, 62.48, 62.36; H, 3.63, 3.61; N, 4.46, 4.47.

The compound of example 105 was prepared by the method of example 104, by utilizing [(9,10-dioxo-9,10-dihydro-phenanthrene-2-carbonyl)-amino]-acetic acid tert-butyl ester as the starting material.

### Example 105:

[(9,10-Dioxo-9,10-dihydro-phenanthrene-2-carbonyl)-amino]-acetic acid:

Orange solid; ¹H NMR (300 MHz, DMSO-d₆) δ12.69 (1H, s), 9.22 (1H, dd, J=6.0, 6.0 Hz), 8.54 (1H, d, J=1.9 Hz), 8.46 (1H, d, J=8.2 Hz), 8.39 (1H, d, J=8.0 Hz), 8.23 (1H, dd, J=1.9, 8.4 Hz), 8.08 (1H, dd, J=1.1, 7.5Hz), 7.82(1H, ddd, J=1.5, 8.0, 8.0 Hz), 7.59 (1H, dd, J=8.0, 8.0 Hz), 3.97 (2H, d, J=5.8 Hz); Anal. Calcd. For C₁₇H₁₁NO₅: C, 66.0; H, 3.58; N, 4.53. Found: C, 66.09, 66.28; H, 3.69, 3.71; N, 4.59, 4.59.

### Example 106

9,10-Phenanthrenedione was purchased from Aldrich Chemical Company, Milwaukee, Wis. and used as received.

Assays for Biological Activity

Method A:

Phosphatase assay using pNPP as substrate:

CD45 enzyme was obtained from BIOMOL (Plymouth Meeting, Pa.). Phosphatase activity was assayed in a buffer containing final concentrations of 25 mM imidazole at pH 7.0, 50 mM NaCl, 2.5 mM ethylenediaminetetraacetic acid (“EDTA”), 5 mM dithiothreitol (“DTT”) and 10 μg/mL bovine serum albumin (“BSA”) using pNPP as a substrate. Compounds were tested in a range from 30 to 0.01 μM, with a final concentration of 1 or 5% dimethylsulfoxide (“DMSO”), depending on the compound solubility. Activity was measured by following the increase in absorbance at 405 nm using a SpectraMax Plus spectrophotometric plate reader (Molecular Devices, Sunnyvale, Calif.).

Method B:

Cytotoxicity Assay:

Calcein-AM (Molecular Probes. Eugene, Oreg.) uptake, as a quantitative measure of cell viability, was used to evaluate the toxic effect of compounds on T cells. Briefly, PBMC were treated for 3-7 days with 3-10 μg/ml PHA, a potent T-cell mitogen, to preferentially expand the T-cell population. (Bradley, Linda M. Cell Poliferation in Selected Methods in Cellular Immunology, Eds. Mishell, B. B. and Shiigi, S. M., W. H. Freeman and Co., San Francisco, 1980.)

The T-cell lymphoblasts were purified by separation over Lymphoprep, plated at 2×10⁵/well in a round bottom 96-well plate containing RPMI with compound and incubated overnight at 37° C. in an incubator containing 5% CO₂. The dilution scheme and culture media were the same as those used in the T-cell proliferation assay. After the incubation period, cells were washed with Dulbecco's phosphate-buffered saline (D-PBS) and incubated with 1 μM Calcein-AM for 30-45 mim in D-PBS as described in the technical sheet provided with The LIVE/DEAD Viability/Cytotoxicity Kit from Molecular Probes. Percent viability was assessed on a fluorescent plate reader (excitation filter 485/20 nm; emission filter 530/25 nm) where the 100% control value is the fluorescence intensity observed in the absence of test compound.

Method C:

Phosphatase assay using lck 10-mer as substrate:

Phosphatase activity was assayed in 96 well plates in a buffer containing final concentrations of 25 mM HEPES at pH 7.2, 5 mM DTT and 10 μg/mL BSA using the lck carboxy-terminal peptide TEGQpYQPQP as the substrate (Cho, H., Krishnaraj, R., Itoh, M., Kitas, E., Bannwarth, W., Saito, H., Walsh, C. T. 1993. Substrate specificities of catalytic fragments of protein tyrosine phosphatases (HPTPb, LAR, and CD45) toward the phosphotyrosylpeptide substrates and thiophosphotyrosylated peptides as inhibitors. Protein Science 2:977-984). Compounds were tested in a range from 30 to 0.01 μM in a final concentration of 5% DMSO. Enzyme was incubated with substrate with or without compound, at room temperature for 1.5 h. At the end of the incubation period, BIOMOL “Green Reagent” (BIOMOL, Plymouth Meeting, Pa.) was added to each well, the plates incubated at room temperature for 30 min and absorbance read at 620 nm.

Method D:

Cell isolation and T cell proliferation assay:

Whole blood was obtained from healthy human blood donors. Peripheral blood mononuclear cells (“PBMC”) were isolated using Lymphoprep density-gradient centrifugation (Nycomed Amersham, Oslo, Norway), washed, counted and resuspended at 2×10⁶ cells/mL in RPMI 1640 medium containing glutamine, 0.1 mg/mL gentamycin and 10% heat inactivated human serum. PBMC were transferred to 96-well plates (2×10⁵ cells/well) containing compound or vehicle control, with the final concentration of DMSO not to exceed 0.3%, and incubated for 1 hour before addition of the activating anti-CD3 antibody, OKT3 (30ng/mL). After 24 hours in culture, the cells were pulsed with [³H]thymidine (1 μCi/well) overnight and harvested the next day onto 96-well Packard GF/C filter plates using a Packard Cell Harvester (Packard Instruments, Meriden, Conn.). The filter plate was dried, the bottom of the plate sealed, 25 μL of Microscint 20 scintillation fluid added to each well, the top of the plate sealed with TopSeal-A, and the plate counted on a Packard TopCount. The data from the TopCount is transferred into Excel 5 (Microsoft, Redmond, Wash.) and formatted for EC₅₀ determination using Prism software (GraphPad Software, San Diego, Calif.).

Table 4 shows the inhibition of CD45 activity in the pNPP asssay and the lck assay certain compounds of the present invention. Inhibition in the T cell proliferation assay, as well as results from T cell cytotoxicity assay are shown.

TABLE 4

| | pNPP IC₅₀ | Ick IC₅₀ | T cell prolif. | |
|---|---|---|---|---|
| Example. No. | (μM) | (μM) | IC_{50 (μM)} | CC₅₀ (μM) |
| 1 | 0.6 | 11 | 3.1 | 20 |
| 2 | 0.5 | 11 | 1.0 | 14 |
| 3 | 0.7 | 17 | 1.1 | 8.2 |
| 4 | 0.2 | 3.8 | 0.1 | 3.5 |
| 5 | 0.3 | 12 | 1.0 | 8.2 |
| 6 | 0.5 | 16 | 1.5 | 12 |
| 7 | 0.5 | 13 | 0.5 | 9 |
| 8 | 0.4 | 15 | 1.4 | 12 |
| 9 | 0.8 | 5.2 | 0.8 | 9.1 |
| 10 | 1.2 | 4.9 | 0.7 | 10 |
| 11 | 2.8 | 11.6 | 6.5 | >30 |
| 12 | 1.3 | 6.0 | 3.0 | 15 |
| 13 | 1.1 | 5.8 | 1.5 | 14 |
| 14 | 12.9 | >30 | 0.8 | 5.3 |
| 15 | 1.0 | 15 | 6.4 | >30 |
| 16 | 3.7 | >30 | 0.8 | 3.6 |
| 17 | 0.8 | >30 | 1.8 | 7.3 |
| 18 | 0.75 | >30 | 1.3 | 8.4 |
| 19 | 4.49 | >30 | 1.6 | 4.4 |
| 20 | 0.99 | >30 | 2.1 | 8.9 |
| 21 | 0.99 | >30 | 1.8 | 4.9 |
| 22 | 3.6 | 9.7 | 0.7 | >30 |
| 23 | 1.1 | 3.0 | 20 | >30 |
| 24 | 0.6 | 2.4 | >30 | >30 |
| 25 | 1.2 | 2.5 | >30 | >30 |
| 26 | 0.7 | 2.3 | >30 | >30 |
| 27 | 0.7 | 3.1 | >30 | >30 |
| 28 | 1.1 | 2.2 | 5 | >30 |
| 29 | 1.1 | 2.5 | 20 | >30 |
| 30 | 0.9 | 1.7 | 14.5 | >30 |
| 31 | 0.6 | 2.5 | 7 | >30 |
| 32 | 0.7 | 2.3 | 10.5 | >30 |
| 33 | 1.1 | 2.5 | 16 | >30 |
| 34 | 0.9 | 1.9 | >30 | ND |
| 35 | 0.9 | 1.4 | >30 | >30 |
| 36 | 1.0 | 2.2 | >30 | >30 |
| 37 | 1.1 | 2.1 | >30 | >30 |
| 38 | 0.7 | 2.2 | >30 | ND |
| 39 | 0.7 | 2.3 | >30 | >30 |
| 40 | 0.6 | 3.9 | 19.3 | >30 |
| 41 | 1.4 | 3.5 | 8.8 | >30 |
| 42 | 0.9 | 3.5 | 3.3 | >30 |
| 43 | 1.9 | 3.8 | 9.0 | >30 |
| 44 | 1.1 | 3.8 | 14.2 | >30 |
| 45 | 1.4 | 3.9 | 5.0 | >30 |
| 46 | 1.0 | 3.8 | 5.5 | >30 |
| 47 | 0.7 | 4.2 | 3.0 | >30 |
| 48 | 0.6 | 5.7 | 11 | >30 |
| 49 | 0.9 | 6.1 | 16 | >30 |
| 50 | 0.7 | 2.8 | 23 | >30 |
| 51 | 1.1 | 4.8 | 14 | >30 |
| 52 | 0.8 | 3.0 | 20 | >30 |
| 53 | 1.0 | 3.7 | 16 | >30 |
| 54 | 0.9 | 3.9 | 20 | >30 |
| 55 | 1.4 | 4.7 | >30 | >30 |
| 56 | 0.8 | 2.3 | 19 | >30 |
| 57 | 1.3 | 3.2 | 21 | >30 |
| 58 | 1.5 | 17.8 | >30 | ND |
| 59 | 1.1 | 5.8 | >30 | ND |
| 60 | 2.0 | 7.9 | 21.4 | ND |
| 61 | 1.8 | 8.5 | 23 | ND |
| 62 | 1.4 | 15.3 | >30 | ND |
| 63 | 0.7 | 2.6 | 1.2 | 8 |
| 64 | 1.1 | >30 | 4.0 | 14 |
| 65 | 1.1 | 3.1 | 0.6 | 15 |
| 66 | 0.9 | 1.8 | 3.5 | 13 |
| 67 | 1.0 | 2.2 | 0.8 | 4 |
| 68 | 1.0 | 3.2 | 1.8 | 7.5 |
| 69 | 0.6 | 4.8 | 4.6 | 14 |
| 70 | 0.4 | 4.7 | 0.5 | 3 |
| 71 | 0.5 | 3.7 | 1.3 | 14 |
| 72 | >30 | ND | ND | ND |
| 73 | 0.5 | 3.0 | 5.0 | 11 |
| 74 | 0.8 | 3.3 | 2.6 | 19 |
| 75 | 0.8 | 10.0 | 2.6 | 13 |
| 76 | 9.3 | >30 | 7.0 | >30 |
| 77 | 0.8 | 2.9 | 1.6 | 9 |
| 78 | 0.7 | 6.9 | 1.3 | 28 |
| 79 | 1.6 | >30 | 0.5 | >30 |
| 80 | 5.9 | 4.9 | 0.7 | 6.5 |
| 81 | 1.2 | 2.5 | 0.2 | 0.9 |
| 82 | 0.5 | 2.9 | 1.1 | 7 |
| 84 | 0.8 | 2.6 | 0.7 | 4 |
| 85 | 1.4 | 2.8 | 0.5 | 4 |
| 86 | >30 | ND | ND | ND |
| 87 | 0.4 | 2.3 | 0.2 | 5 |
| 88 | 3.74 | 10.5 | 0.2 | 17 |
| 89 | 0.8 | 2.9 | 0.2 | 9 |
| 90 | 0.4 | 3.2 | 1.0 | 18 |
| 91 | 0.4 | 4.7 | 0.5 | 9 |
| 92 | 2.4 | 5.4 | 1.6 | 10 |
| 93 | 0.5 | 3.9 | 1.3 | 10 |
| 94 | 4.1 | 5.0 | 0.5 | 8 |
| 95 | >30 | >30 | >30 | 30 |
| 96 | 0.37 | 3.1 | 0.6 | 5 |
| 97 | 1.0 | 3.3 | >30 | >30 |
| 98 | 1.0 | 2.4 | >30 | ND |
| 99 | 0.5 | 4.9 | 0.2 | 2.8 |
| 100 | 0.8 | 4.2 | 0.8 | 3.5 |
| 101 | 0.8 | 2.9 | 1.1 | 4.5 |
| 102 | 0.6 | 3.4 | 0.3 | 2.5 |
| 103 | 0.6 | 4.9 | 0.4 | 10 |
| 104 | 0.5 | 3.4 | 6.5 | >30 |
| 105 | 0.8 | 2.2 | 4.4 | >30 |
| 106 | 0.7 | 3.1 | 0.3 | 1.3 |

We claim:

1. Any compound in accord with structural diagram I, 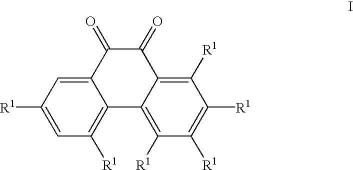
or tautomers thereof or pharmaceutically-acceptable salts thereof,
wherein:
R¹ at each occurrence is independently selected from hydrogen, halogen, NH₂, NO₂, NH—CO—R², CO—NH—R², Ar, (CH₂)ₙCH(COOH)R³ COR³, NHCOCH₂CH(COOH)NHR⁴ and N(R⁵)₂,
wherein:
R² is selected from (C₁-C₄)alkyl, (C₁-C₈)alkylCOOR⁶ and phenyl, wherein phenyl moieties of R² can be substituted at one, two or three positions with a moiety selected from halogen, NO₂ and CF₃, alkyl moieties of R² can be substituted with halogen, and R⁶ is selected from hydrogen and (C₁-C₄)alkyl;
Ar at each occurrence is independently selected from groups in accord with the following structural diagrams, 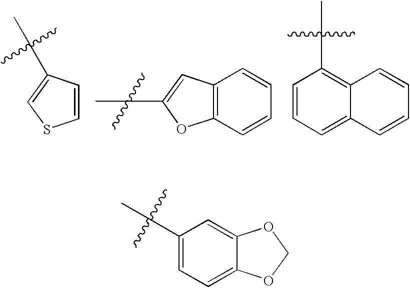
or from phenyl which can be substituted with a moiety selected from halogen, (C₁-C₄)alkyl, O-(C₁-C₄)alkyl and halo(C₁-C₄)alkyl;
R³ at each occurrence is an N-linked oligopeptide selected from GQ-N-iBu, GQPQP, QQPQP, EQPQP, GEPQP, QEPQP, GQGQP, QQGQP, EQGQP, GEGQP, QEGQP, QQPEG, GQGEP, GQPQG, QQPQG, GEPQG, GQPEG, GGPEG, EGPEG, RGPEG, GEPEG, EEPEG, REPEG, GRPEG, ERPEG, RRPEG, GGPRG, EGPRG, RGPRG, GEPRG, EEPRG, REPRG, GRPRG, ERPRG and RRPRG,
R⁴ at each occurrence is a C-linked N-acetyl-oligopeptide selected from Q, EGQ, ATEGQ, TATEGQ, and FTATEGQ, and
R⁵ at each occurrence is selected from hydrogen and tosyl;
with the proviso that said compound is not 2-amino-5-nitro-phenanthrene-9,10-dione; 2,5-diamino-phenanthrene-9,10-dione; 2-amino-phenanthrene-9,10-dione; 3-amino-phenanthrene-9,10-dione; 2-nitro-phenanthrene-9,10-dione; 4-nitro-phenanthrene-9,10-dione; 2,7-dinitro-phenanthrene-9,10-dione; 2,5-dinitro-phenanthrene-9,10-dione; 2-bromo-phenanthrene-9,10-dione; 9,10-dioxo-9,10-dihydro-phenanthrene-2-carboxylic acid; 9,10-dioxo-9,10-dihydro-phenanthrene-3-carboxylic acid; 3-nitro-phenanthrene-9,10-dione; 3-acetyl-phenanthrene-9,10-dione; 2-acetyl-phenanthrene-9,10-dione, or 9,10-phenanthrenedione.

2. A compound according to claim 1, in accord with structural diagram II, 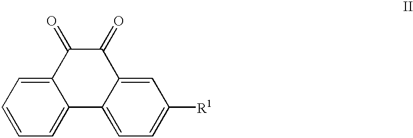
wherein:
R¹ is N(R⁵)₂, where R⁵ is selected from hydrogen and tosyl with the proviso that at least one R⁵ moiety is tosyl.

3. A compound according to claim 1, in accord with structural diagram III, 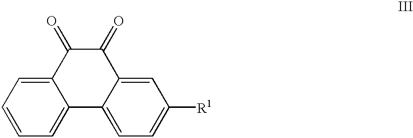
wherein:
R¹ is Ar and Ar is selected from groups in accord with the following structural diagrams: 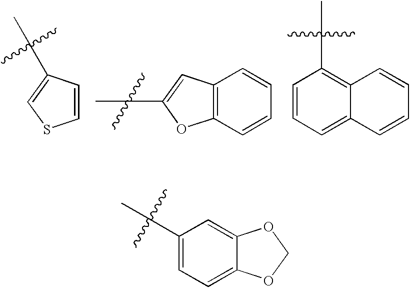
or from phenyl which can be substituted with a moiety selected from halogen, (C₁-C₄)alkyl, O-(C₁-C₄)alkyl and halo(C₁-C₄)alkyl.

4. A compound according to claim 3, wherein Ar is phenyl substituted with perfluoromethyl.

5. A compound according to claim 1, in accord with structural diagram IV 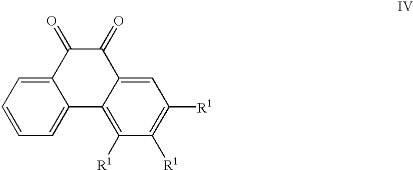
wherein:
R¹ is selected from hydrogen, halogen, NO₂, NH₂, (CH₂)ₙCH(COOH)R³ and COR³ where R³ is an N-linked peptide selected from GQ-N-iBu, GQPQP, QQPQP, EQPQP, GEPQP, QEPQP, GQGQP, QQGQP, EQGQP, GEGQP, QEGQP, QQPEG, GQGEP, GQPQG, QQPQG, GEPQG, GQPEG, GGPEG, EGPEG, RGPEG, GEPEG, EEPEG, REPEG, GRPEG, ERPEG, RRPEG, GGPRG, EGPRG, RGPRG, GEPRG, EEPRG, REPRG, GRPRG, ERPRG and RRPRG, with the proviso that no more than one R¹ moiety is other than hydrogen.

6. A compound according to claim 1, wherein R¹ is selected from hydrogen, NH₂, and NHC(O)(CH₂)ₙCOOCH₃ where n is an integer selected from the range 1 to 8.

7. A method for treating immunologically-related diseases, autoimmune disorders and organ graft rejection comprising administering to a subject suffering therefrom an effective amount of a compound in accord with structural diagram I, 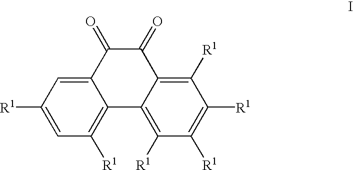
or tautomers thereof or pharmaceutically-acceptable salts thereof,
wherein:
R¹ at each occurrence is independently selected from hydrogen. halogen, NH₁₂, NO₂, NH—CO—R², CO—NH—R², Ar, (CH₂)ₙCH(COOH)R³ COR³, NHCOCH₂CH(COOH)NHR⁴ and N(R⁵)₂,
wherein:
R² is selected from (C₁-C₄)alkyl, (C₁-C₈)alkylCOOR⁶ and phenyl, wherein phenyl moieties of R² can be substituted at one, two or three positions with a moiety selected from halogen, NO₂ and CF₃, alkyl moieties of R² can be substituted with halogen, and R⁶ is selected from hydrogen and (C₁-C₄)alkyl;
Ar at each occurrence is independently selected from groups in accord with the following structural diagrams, 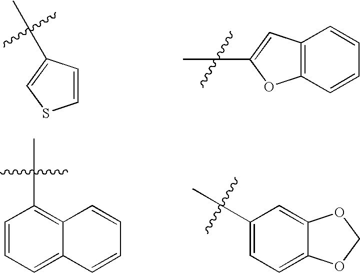
or from phenyl which can be substituted with a moiety selected from halogen, (C₁-C₄)alkyl, O-(C₁-C₄)alkyl and halo(C₁-C₄)alkyl;
R³ at each occurrence is an N-linked oligopeptide selected from GQ-N-iBu, GQPQP, QQPQP, EQPQP, GEPQP, QEPQP, GQGQP, QQGQP, EQGQP, GEGQP, QEGQP, QQPEG, GQGEP, GQPQG, QQPQG, GEPQG, GQPEG, GGPEG, EGPEG, RGPEG, GEPEG, EEPEG, REPEG, GRPEG, ERPEG, RRPEG, GGPRG, EGPRG, RGPRG, GEPRG, EEPRG, REPRG, GRPRG, ERPRG and RRPRG,
R⁴ at each occurrence is a C-linked N-acetyl-oligopeptide selected from Q, EGQ, ATEGQ, TATEGQ, and FTATEGQ, and
R⁵ at each occurrence is selected from hydrogen and tosyl.

8. A pharmaceutical composition comprising an effective amount of a compound according to any one of claims 1 to 6, and a pharmaceutically-acceptable excipient or diluent.
